(19) United States
(12) Patent Application Publication
(10) Pub. No.: US 2024/0331856 A1
(43) Pub. Date: Oct. 3, 2024

(54) SYSTEM AND METHOD TO RECOMMEND TOOL SET FOR A ROBOTIC SURGICAL PROCEDURE

(71) Applicant: Intuitive Surgical Operations, Inc., Sunnyvale, CA (US)

(72) Inventors: Ryan W. Shaw, San Jose, CA (US); Joseph M. Fridlin, Chesterfield, MT (US)

(73) Assignee: Intuitive Surgical Operations, Inc., Sunnyvale, CA (US)

(21) Appl. No.: 18/617,512

(22) Filed: Mar. 26, 2024

Related U.S. Application Data
(60) Provisional application No. 63/454,896, filed on Mar. 27, 2023.

(51) Int. Cl. G16H 40/63

(52) U.S. Cl. CPC G16H 40/63

(57) ABSTRACT

A computer-implemented method is provided to receive skill data and patient data, the skill data indicative of a skill level associated with a surgeon, and the patient data corresponding to a patient, determine, using a model trained with machine learning and receiving as input the skill data and the patient data, tool data indicative of a recommended tool, and cause, based on the tool data, an indication to be presented to a user to install the recommended tool onto the computer-assisted robotic system.

### CROSS-REFERENCE TO RELATED PATENT APPLICATIONS

This application claims the benefit of priority under 35 U.S.C. § 119 to U.S. Provisional Patent Application Ser. No. 63/454,896, entitled “SYSTEM AND METHOD TO RECOMMEND TOOL SET FOR A ROBOTIC SURGICAL PROCEDURE,” filed Mar. 27, 2023, the contents of such application being hereby incorporated by reference in its entirety and for all purposes as if completely and fully set forth herein.

### BACKGROUND

Minimally invasive robotic surgical procedures can involve a team of medical practitioners that include a surgeon and one or more medical professional members of a care team who provide medical and technical support. A robotic surgical procedure is preceded by extensive planning. The surgeon studies a patient's medical records and develops a plan for the procedure. A care team sets up the operating room to perform the procedure according to the surgical plan. The set up may include physically arranging positions of components of surgical system, retrieving surgical tools from inventory, and preparing the tools for use during the surgical procedure.

Setting up surgical tools for a surgical procedure can be timely and costly. Different surgeons may use different combinations of tools for the same procedure and individual surgeons may not use the same tool set from one instance of a surgical procedure to the next. As a result, care teams may open up disposable inventory that is not needed, leading to waste medical inventory, and additional cost for a surgical procedure. In view of this challenge, care teams may maintain lists of instruments that different physicians were expected to use for various procedures. These lists can be based upon instruments picked out by physicians and are can be referred to as “pick lists.” However, pick lists easily could become out of date as surgeons change preferences as to the assortment of instruments to use for a procedure. Electronic Medical Records (EMRs) have enabled the digitization of pick lists making them more easily accessible to care teams over a network and easier to update to keep them current. In an example medical record system, a surgeon can create an electronic ‘card’ within that specifies the surgeon's pick list for a surgical procedure, and a care team can access a surgeon's electronic card containing a pick list over a network.

Nevertheless, EMRs still rely on its users to regularly update the pick lists, which are generally accessible over local networks. Further, the optimal set of tools for use by a surgeon for a medical procedure may depend upon a variety of factors that are not within the personal knowledge or experience of the surgeon or the care team. Accordingly, there exists a need for improved guidance to the selection of tool sets for use during medical procedures.

### SUMMARY

In one aspect, a system for recommending a tool for a computer-assisted robotic system is provided. The system can include a non-transitory memory and one or more processors. The system can receive skill data and patient data, the skill data indicative of a skill level associated with a surgeon, and the patient data corresponding to a patient. The system can determine, using a model trained with machine learning and receiving as input the skill data and the patient data, tool data indicative of a recommended tool. The system can cause, based on the tool data, an indication to be presented to a user to install the recommended tool onto the computer-assisted robotic system.

In another aspect, a method for recommending a tool for a computer-assisted robotic system is provided. The method can include receiving, by a processor, skill data and patient data, the skill data indicative of a skill level associated with a surgeon, and the patient data corresponding to a patient. The method can include determining, by the processor using a model trained with machine learning and receiving as input the skill data and the patient data, tool data indicative of a recommended tool. The method can include causing, by the processor and based on the tool data, an indication to be presented to a user to install the recommended tool onto the computer-assisted robotic system.

In another aspect, a non-transitory computer readable medium can include one or more instructions stored thereon and executable by a processor. The processor can receive skill data and patient data, the skill data indicative of a skill level associated with a surgeon, and the patient data corresponding to a patient. The processor can determine, using a model trained with machine learning and receiving as input the skill data and the patient data, tool data indicative of a recommended tool. The processor can cause, based on the tool data, an indication to be presented to a user to install the recommended tool onto the computer-assisted robotic system.

In another aspect, a computer-implemented method for generating and presenting display of tool selection guidance for a robotic surgical procedure is provided. A current surgical procedure data set is received that includes skill data of a current surgeon and patient health data of a current patient for a current surgical procedure. A tool set recommendation engine is used to determine recommended tool set information based upon the current surgical procedure data set.

In another aspect, a system for generating and presenting display of tool selection guidance for a robotic surgical procedure is provided. The system includes a processing unit and a memory storing instructions that, upon execution by the processing unit, causes the system to perform operations. The operations include receiving a current surgical procedure data set that includes skill data of a current surgeon and patient health data of a current patient for a current surgical procedure. The operations further include determining a recommended tool set information based upon the current surgical procedure data set.

### BRIEF DESCRIPTION OF DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for simplicity and clarity and does not in itself dictate a relationship between the various embodiments and/or configurations discussed.

FIG. 1 is an illustrative plan view of an example minimally invasive robotic surgical system.

FIG. 2 is an illustrative perspective view of an example user control unit of the robotic surgical system of FIG. 1.

FIG. 3 is an illustrative perspective view of an example manipulator unit of the robotic surgical system of FIG. 1.

FIG. 4 is an illustrative side elevation view of an example surgical tool for use with the robotic surgical system of FIG. 1.

FIG. 5 is an illustrative drawing representing an example robotic surgical tool set recommendation system according to some embodiments.

FIG. 6 is an illustrative flow diagram representing an example method of operation of the robotic surgical tool set recommendation system of FIG. 5.

FIG. 7 is an illustrative flow diagram representing a machine learning-based method of operating the tool set recommendation engine of FIG. 5.

FIG. 8A is an illustrative drawing representing a method of training a machine learning model of the machine learning-based method of FIG. 7.

FIG. 8B is an illustrative drawing representing a method of using a trained machine learning model of the machine learning-based method of FIG. 7.

FIG. 9 is an illustrative flow diagram representing an example pattern-based method of operating the tool set recommendation engine of FIG. 5.

FIG. 10 is an illustrative data flow diagram representing an example use of the pattern-based method of FIG. 9.

FIG. 11 is an illustrative flow diagram representing a method of operating the tool set recommendation engine of FIG. 5 to use intra-operative surgical procedure data to produce an intra-operative tool set recommendation.

FIG. 12A is an illustrative drawing representing an example first user interface (UI) screen display produced at a display block.

FIG. 12B is an illustrative drawing representing an example second UI screen display produced at the display block.

FIG. 12C is an illustrative drawing representing an example third UI screen display produced at the display block.

FIG. 12D is an illustrative drawing representing an example fourth UI screen display produced at the display block.

FIG. 12E is an illustrative drawing representing an example fifth UI screen display produced at a display block.

FIG. 12F is an illustrative drawing representing an example sixth UI screen display produced at a display block.

FIG. 12G is an illustrative drawing representing an example seventh UI screen display produced at a display block.

FIG. 13 illustrates components of a computing machine, according to some example embodiments.

FIG. 14 illustrates an example method of recommending a tool for a computer-assisted robotic system, according to some example embodiments.

FIG. 15 illustrates an example method of presenting a recommendation for a tool for a computer-assisted robotic system, according to some example embodiments.

### DETAILED DESCRIPTION

The present disclosure is directed to systems and methods for recommending a tool set for a surgical procedure to be performed using a robotic surgical system. In an embodiment, a tool set recommendation system receives recorded surgical procedure information such as surgical procedure type, surgeon skill level, patient health features, and/or surgical outcome corresponding to previous surgical procedures. An example tool set recommendation system uses the recorded surgical procedure information together with information about a current surgical procedure to generate a tool set recommendation for the current surgical procedure. The tool set recommendation can include pre-operative and intra-operative tool set recommendations. The term ‘tool’, ‘surgical tool’ as used herein refers to any instrumentality or device introducible into the human body. The term may refer to any location on the tool. For example, the term can refer to the tip of the same, the body of the same and any combination thereof.

### A. Robotic Surgical System:

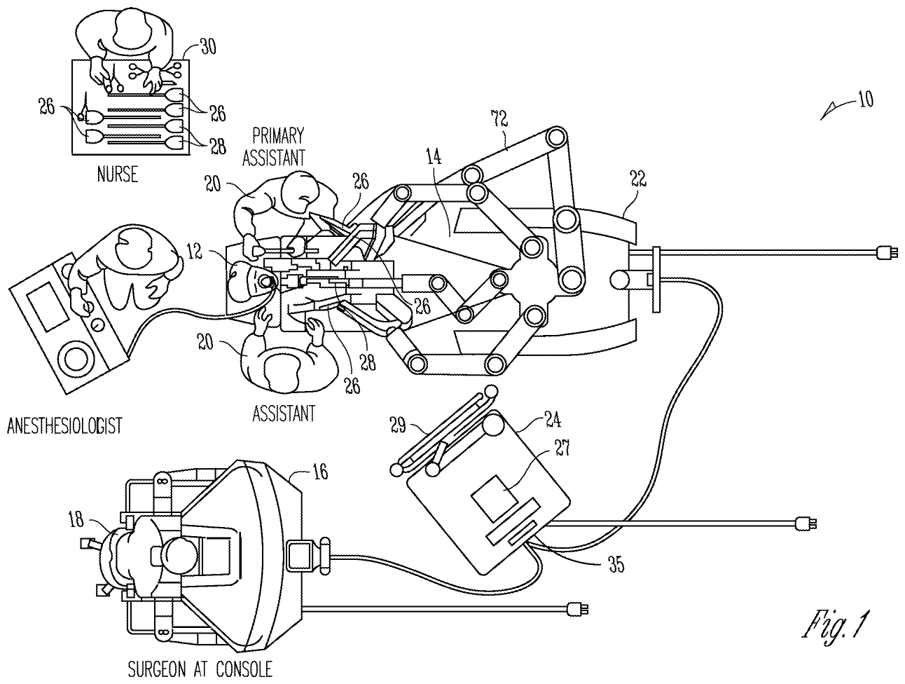

FIG. 1 is an illustrative plan view of an example minimally invasive robotic surgical system (RSS) 10 for performing a minimally invasive surgical procedure on a patient 12 shown lying on an operating table 14. The system includes a user control unit 16 for use by a surgeon 18, one or more manipulator units 22, and an auxiliary unit computer processing subsystem 24. The manipulator units 22 can manipulate at least one robotic surgical tool 26 through a minimally invasive incision in the body or a natural body orifice of the patient 12 while the surgeon 18 views the surgical site through the user control unit 16. An image of the surgical site can be obtained by an endoscope 28, such as a stereoscopic endoscope, which is a tool that may be positioned using a manipulator unit 22. The endoscope 28 includes an imaging device that can capture and store images of a surgical site within a patient's anatomy. In some embodiments, stereoscopic images may be captured, which allow the perception of depth during a surgical procedure. The number of surgical tools 26 used at one time will generally depend on the surgical procedure and the space constraints within the operative site, among other factors. If it is necessary to change one or more of the robotic surgical tools 26 being used during a procedure, an assistant 20 may remove the robotic surgical tool 26 from a manipulator unit 22 and replace it with another robotic surgical tool 26 from a tray 30 in the operating room.

The auxiliary unit 24 includes a computer processing subsystem 27 that includes one or more electronic processor devices for processing the images of a surgical site captured using the endoscopic imaging device 28 for display to the surgeon 18 through the user console 16. In certain embodiments, the computer processing subsystem 27 may be a separate unit coupled to the auxiliary unit 24 and may be centralized or distributed. The computer processing system 27 includes a logic unit, such as one or more electronic processor circuits, and a memory that stores instructions carried out by the logic unit.

The auxiliary unit 24 includes a display screen 29 and user input devices 35 which can include mouse and keyboard and/or a touchscreen interface. The auxiliary unit 24 can control the display screen 29 to display information viewable by a surgical care team within the operating room. The displayed information can include tool set recommendations and user tool set preferences. It is contemplated that tool set recommendations may be input to the auxiliary unit 24 over an electronic data channel, such as a network connection (not shown), by a tool set recommendation engine 502 described more fully below. The auxiliary unit 24 can control display of the tool set recommendations at the display screen 29. An operator may input user (e.g., surgeon) tool set preferences to the auxiliary unit 24 via an electronic device such as network console, personal computer, handheld communication device, or other network-connected device, over a network connection (not shown). The auxiliary unit 24 can control configuration of the robotic surgical system 10 to use the selected tool set selection. During a surgical procedure, the auxiliary unit 24 can control the display screen 29 also can display surgical site images, from within a patient's anatomy, for viewing by a surgical team within the operating room that are simultaneously viewed by a surgeon via a viewer display 31.

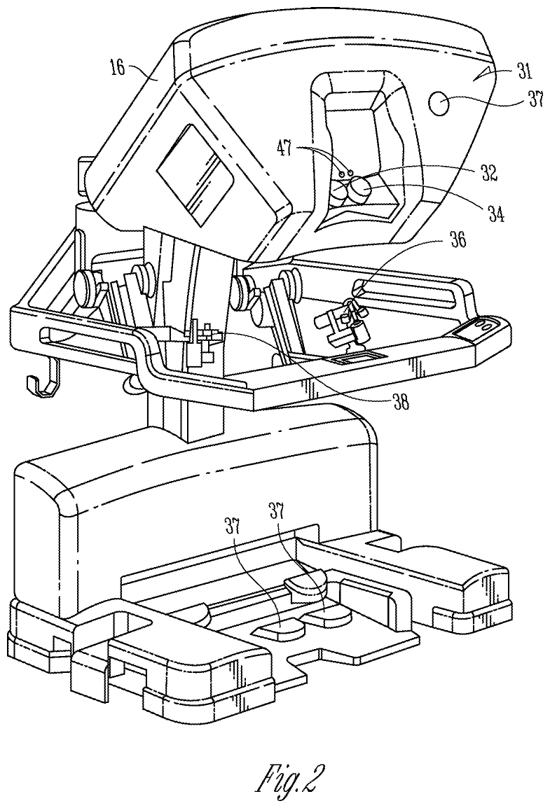

FIG. 2 is a perspective view of an example user control unit 16 of the robotic surgical system 10. The example control unit 16 includes a viewer display 31 that includes a left eye display 32 and a right eye display 34 for presenting the surgeon 18 with a coordinated stereoscopic view of the surgical site, produced using the computer processing subsystem 27, which enables depth perception. The control unit 16 also can be equipped with microphone 37 and speaker system (not shown) to enable a surgeon to exchange audio messages with the control unit 16 and with members of a surgical team. The control unit 16 further includes one or more hand-operated control input devices 36, 38 to receive larger-scale hand control movements. One or more robotic surgical tools 26 installed for use at s corresponding manipulator unit 22 are operatively coupled to move in relatively smaller-scale distances that match a surgeon 18's manipulation of the one or more control inputs 36, 38. In an example system, each control input device 36, 38 is operatively coupled to control a robotic surgical tool. For example, a first control input device 36 can be operatively coupled to control a first robotic surgical tool and a second control input device 38 can be operatively coupled to control a second robotic surgical tool.

During a surgical procedure, multiple different surgical tools can be available at an instrument tray 30, for example, for installation to the manipulator unit 22 for user control via the control unit 16. An example control unit 16 includes a clutch mechanism (not shown) operable by a surgeon to change correspondence between control input devices device 36, 38 and surgical tools 26. For example, a manipulator unit 22 may be coupled to three surgical robotic tools 26 and the clutch mechanism can be used to couple the control input devices device 36, 38 to different pairs of the tools 26. The control input devices 36, 38 may provide the same mechanical degrees of freedom (DOF) as their associated robotic surgical tools 26 to provide the surgeon 18 with telepresence, or the perception that respective control input devices 36 are operatively coupled to and integral with the corresponding respective controlled robotic surgical tools 26 so that the surgeon has a keen sense of directly controlling the robotic surgical tools 26.

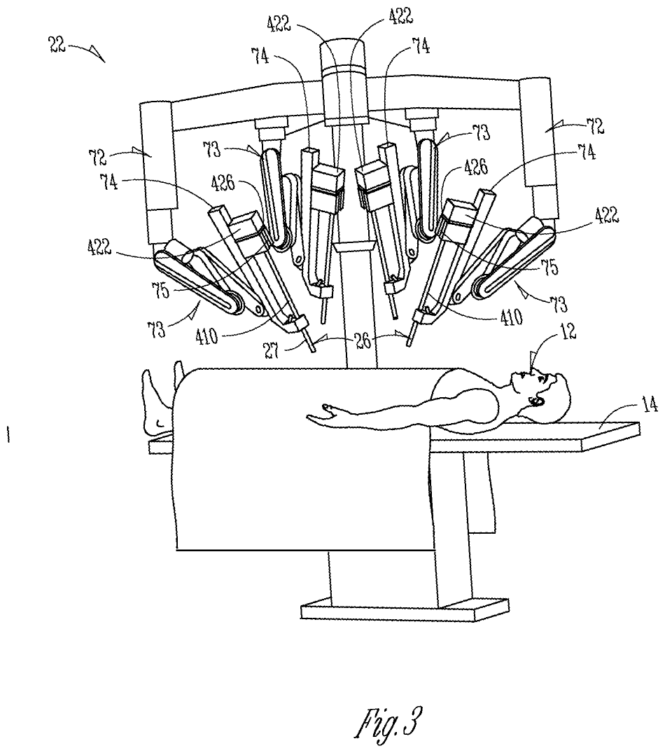

FIG. 3 is a perspective view of an example manipulator unit 22 of the robotic surgical system 10. The manipulator unit 22 includes four manipulator support structures 72. Each manipulator support structure 72 includes articulated support structures 73 that are pivotally mounted end-to-end and a pivotally mounted support spar 74. A respective surgical instrument carriage 75, which includes actuators to control instrument motion, is mounted at each support spar 74. Each robotic surgical tool 26 is detachably coupled to a carriage 75 mounted to a spar 74. A mechanical adapter input interface 426 located between each carriage 75 and each robotic surgical tool includes drive inputs (not shown) driven by actuators within the carriage 75 that configured to couple rotational torque produced by the actuators to drive elements of the robotic surgical tool. A surgeon manipulates the control input devices 36, 38 to control robotic surgical tool's end effector. An input provided by a surgeon or other medical person to a control input device 36 or 38 (an “input” command) is translated into a corresponding action by the robotic surgical tool 26 (a corresponding “surgical instrument” response) through actuation of one or more remote actuators.

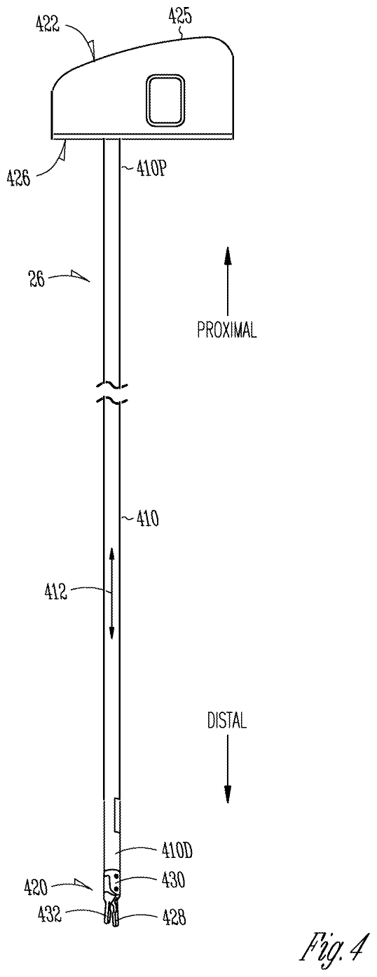

FIG. 4 is an illustrative side elevation view of an example surgical tool for use with the robotic surgical system 10. The example surgical tool 26 includes a shaft defining an internal bore and including a first end portion 410P and a second end portion 410D. A mechanical control mechanism 422 is coupled to the first end portion of the shaft 410. The mechanical control mechanism 422 includes a drive assembly that includes drive elements (not shown) enclosed within a housing 425 used to control movement of a movable component 428 and a wrist 430 located at the second end portion 410D of the shaft 410. The moveable component 428 can include an end effector used to carry out a therapeutic, diagnostic, or an imaging surgical function, or any combination of these functions. For example, the movable component 428 can include any one of a variety of end effectors, such as tissue grasping jaws, a needle driver, shears, a bipolar cauterizer, a tissue stabilizer or retractor, a clip applier, an anastomosis device, an imaging device (e.g., an endoscope or ultrasound probe), and the like. Some surgical instruments used with embodiments further provide an articulated support (sometimes referred to as a “wrist”) for the end effector so that the position and orientation of the end effector can be manipulated with one or more mechanical DOFs in relation to the instrument's shaft 410. The wrist 430 is coupled at the distal end portion 410D of the shaft, proximal of the movable component 428, allows the orientation of the moveable component 428 to be manipulated with reference to the elongated shaft 410. The moveable component 428 may include a force sensor 432 that can be configured to sense force imparted to tissue and/or that can be configured to provide haptic feedback at control inputs 36, based upon force imparted at the moveable component 428 during contact with anatomical tissue.

### B. Robotic Tool Recommendation System:

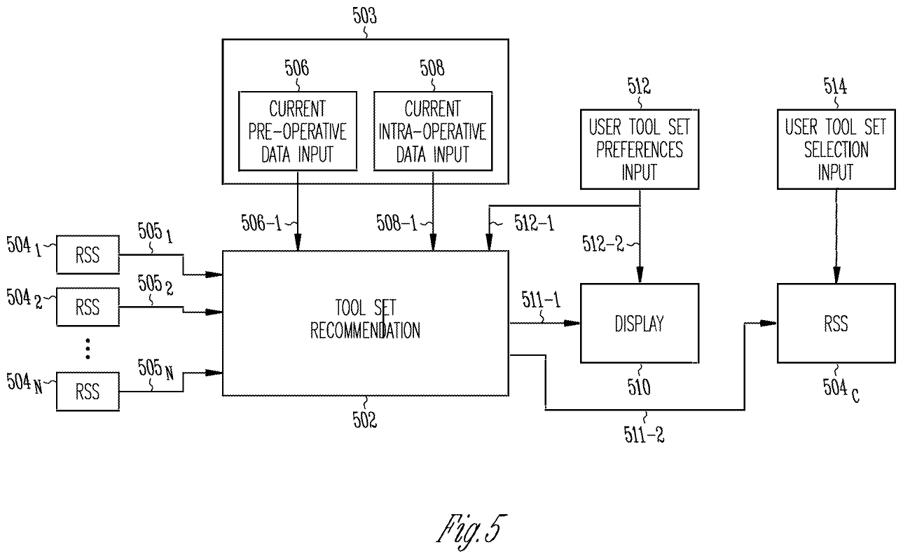

FIG. 5 is an illustrative drawing representing an example robotic surgical tool set recommendation system 500 according to some embodiments. The system 500 includes a tool set recommendation engine 502 and one or more surgical robotic surgical systems (RSSs) 504₁-504ₙ. The tool set recommendation engine 502 is coupled to receive current surgical procedure data 503, which can include current pre-operative surgical procedure data 506 and/or current intra-operative surgical procedure data 508. The tool set recommendation system 502 also is configured to receive user tool set preference data 512. The system 500 includes a display block 510 that is configured to receive and display tool set recommendation information produced at line 511-1 using the tool set recommendation engine 502. The system 500 includes a current robotic surgical system (RSS) 504_{C}, coupled to be configurable based upon tool set recommendation information produced at line 511-2 using the tool set recommendation engine 502.

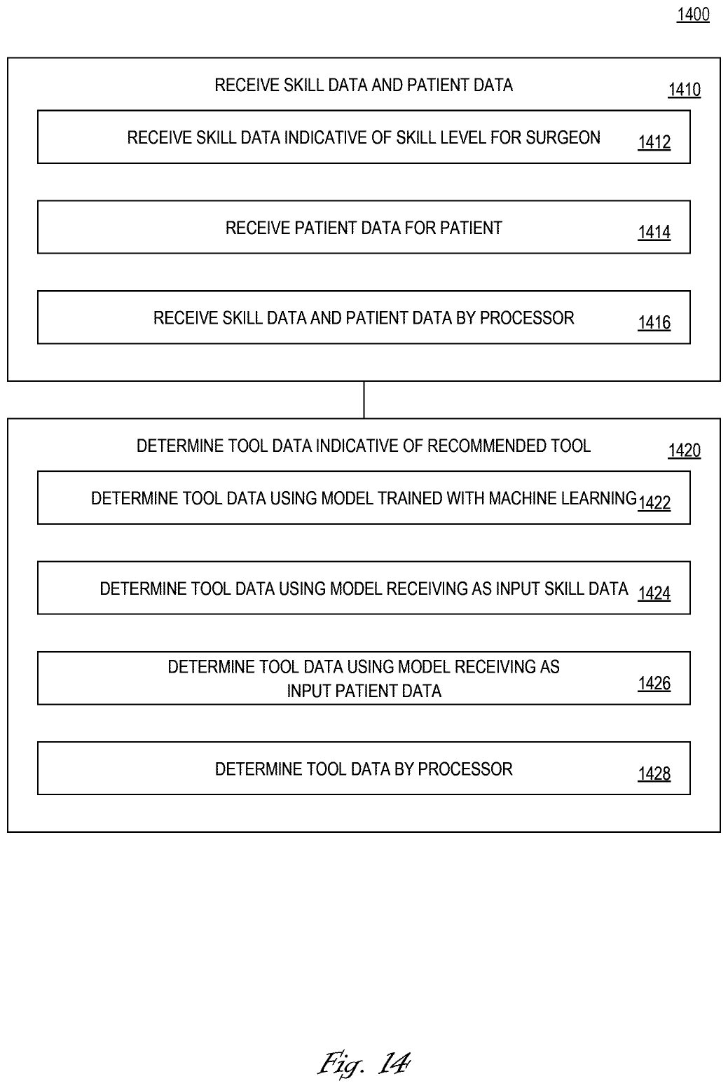

An example tool recommendation engine 502 can be implemented using one or more computing machines 1600 described below with reference to FIG. 13. It is contemplated that the tool set recommendation engine 502 can be implemented as computing machine 1600 implemented at a computer processing subsystem 27 at an instance of the robotic surgical system 10 described above. Alternatively, the tool recommendation engine 502 can be implemented as a computing machine 1600 implemented at a centralized computing system. Each of the multiple robotic surgical systems 504₁-504ₙ can be an instance of the example robotic surgical system 10 described above coupled via network connections (not shown) to the tool recommendation engine 502. Moreover, the current RSS 504_{C} can be one of the one or more RSSs 504₁-504ₙ.

An example tool set recommendation engine 502 receives one or more surgical procedure data sets, indicated by arrows 505₁ to 505ₙ, from each of one or more of the robotic surgical systems 504₁-504ₙ that provide information about a plurality of previously performed surgical procedures. The tool set recommendation engine 502 also can receive the current surgical procedure data 503 corresponding to a current surgical procedure. The current procedure data 503 can include current pre-operative data 506 or current intra-operative data 508 or a combination of both. The tool set recommendation engine 502 is configured to produce a tool set recommendation for a current surgical procedure based upon the current surgical procedure data 503. As explained below, an example tool set recommendation engine 502 can include a machine learning model that is trained based upon a plurality of previous surgical procedure data sets 505₁ to 505ₙ. The trained machine learning model can be used to produce the tool set recommendation for a current surgical procedure based upon the current surgical procedure data 503 (i.e., current pre-operative, current intra-operative, or a combination thereof).

An example tool set recommendation engine 502 includes inputs operable to receive the current pre-operative data 506 at line 506-1 and optionally, to receive the current intra-operative data 508 at line 508-1. The inputs to receive current intra-operative data 508 may include electronic connections to one or more of an imaging device 28, a microphone 37, or a force sensor 432 of the current RSS 504_{C} to receive current intra-operative surgical procedure data 508. It will be appreciated that in some surgical procedures, current intra-operative data 508 is used only for special circumstances that may require modification of an original recommendation based upon current pre-operative data 506, and therefore, current intra-operative data 508 may not be required in all circumstances.

An example tool set recommendation engine 502 also includes inputs operable to optionally receive user tool set preference information 512 to the tool set recommendation block at line 512-1 and to the display block 510 at line 512-2. An example tool set recommendation engine 502 can be configured to prioritized to recommend a user's preferred tools if they are among alternative tools suitable for a task. A user's tool set preferences may include tools used previously by a user during a prior surgical procedure that is like the current surgical procedure and/or a tool set provisionally proposed for use by a user before consideration of a current tool set recommendation. A surgeon's tool set preferences are sometimes referred to as a surgeon's “pick list”. A user's preferences can be input via an input device 35 or a network connection to a user device such as a mobile phone (not shown), at the auxiliary unit 24.

The display block 510 can be implemented to include a display co-located at the current RSS 504_{C}, which can be one of the surgical robotic surgical systems 504₁-504ₙ. The display block 510 can include a display screen 29 of the current RSS 504_{C} that is viewable by multiple surgical team members located at an operating room in which the current RSS 504_{C} is located. The display block 510 can be implemented to also include a display screen viewable by a surgeon at a viewer display 31 of the current RSS 504_{C}.

Lines 511-1, 511-2 indicate tool set recommendation information produced using tool set recommendation engine 502 that is provided to the display block 510 and that can be used to cause visual display of a tool set recommendation produced using the tool set recommendation engine 502. Line 512-1 indicates user tool set preference information that can be provided to the display screen block 510 to display user preferences. It is contemplated that the display block 510 can be used to display both a tool set recommendation produced using the tool set recommendation engine 502 and a user's tool set preferences 512.

Line 511-1 indicates tool set recommendation information produced using tool set recommendation engine 502 that is provided to the current RSS 504_{C} and that can be used to configure the current RSS 504_{C}, based upon user (e.g., a surgeon) selection of a recommendation indicated on the display screen block 510.

User tool set selection 514 can be input via an input device 35 or a network connection to a user device (not shown), at the auxiliary unit 24, coupled to cause configuration of the current RSS 504_{C} to use a user's selected tool set. As explained above, a user's selected tool set may include tools from a tool set recommended by the tool set recommendation engine 502, and/or tools from a user's tool set preferences, which can be displayed at display block 510.

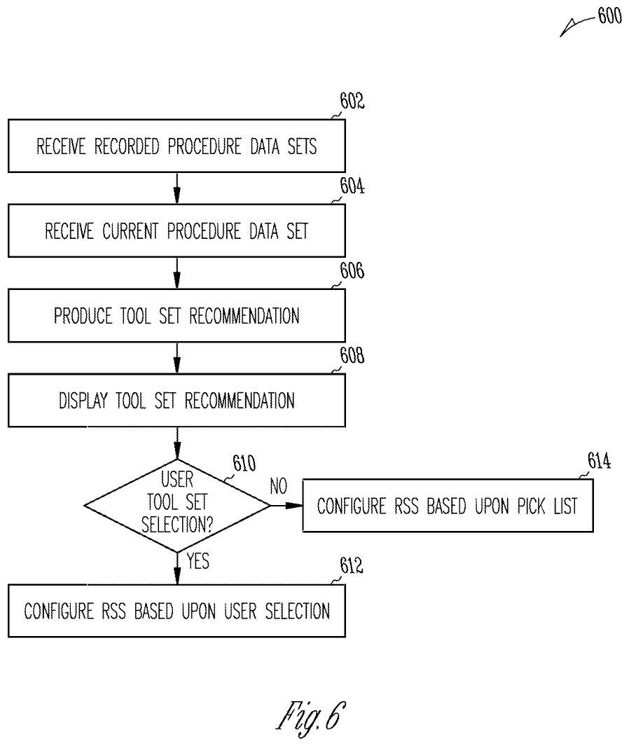

FIG. 6 is an illustrative flow diagram representing an example method 600 of operation of the robotic surgical tool set recommendation system 500 of FIG. 5. The one or more computing machines 1600 used to implement the example tool set recommendation engine 502, can be configured with instructions to perform the operations of the example method 600. Operation 602 receives recorded surgical procedure data sets 505₁-505ₙ produced based upon surgical procedures performed at one or more of RSSs 504₁-504ₙ. As explained more fully below, one or more surgical procedures may be performed at each of the one or more RSSs 504₁-504ₙ. Operation 604 receives current procedure data, which may include pre-operative data and/or intra-operative data. Operation 606 produces a tool set recommendation based upon the surgical procedure data sets and the current pre-operative data and/or current intra-operative data. Operation 606 may provide a tool set recommendation in part before performance of the current surgical procedure, based at least in part upon current pre-operative data. Operation 606 may provide a tool set recommendation during performance of the current surgical procedure, based at least in part upon current intra-operative data. Operation 608 causes display of a tool set recommendation at a display screen 29 and/or 31. Operation 608 may display a tool set recommendation in part before and in part during performance of the current surgical procedure. Operation 610 determines whether a user tool set selection is received. For example, a surgeon may select tools from the tool set recommendation received from the tool set recommendation engine 502 or from a surgeon's pick list or from a combination of both. A user (e.g., a surgeon) can communicate tool set preferences via a mobile device pre-operatively for pre-operation recommendation, or via the surgeon console intra-operatively for an intra-operative recommendation. In response to a determination at operation 610 that user selection is received, operation 612 configures the RSS 504_{C} to use tools during a current surgical procedure based upon a user's selection of tools from among the tool set recommendation and/or the user's pick list. In an example embodiment, in the absence of a user selection, operation 614 configures the RSS 504_{C} to use tools during a current surgical procedure, based upon the user's (e.g., the surgeon's) pick list.

It is contemplated that 612, 614 can configure an RSS 504_{C} in a number of ways. Operations 612, 614 can configure an RSS 504_{C} to constrain kinematics such as to restrict of range of motion or rate of motion of a surgical tool during a surgical stage and or event based upon surgeon skill level. Operations 612, 614 can configure an endoscope position relative to a surgical scene and/or other surgical instruments. Operations 612, 614 can configure an RSS 504_{C} to present images on a screen display 510 from previous similar surgical procedures or from current pre-operative images (e.g., MRI, CT) together with real-time images of a surgical scene. Operations 612, 614 can configure RSS 504_{C} to take sensor measurements such as proximity of a surgical instrument to patient tissue. Operations 612, 614 can configure RSS 504_{C} for registration of a tool with a patient's anatomical features and/or dimensions. Operations 612, 614 can configure RSS 504_{C} to scale rate of motion of a tool during a stage and/or event. Operations 612, 614 can configure RSS 504_{C} to configure articulated support structures 73 and/or support spars 74, and/or table angle.

As a further example, operations 612, 614 can configure RSS 504_{C} to make intra-operative recommendations as to which tool to use for different tissue types. For example, extra care generally is required when using a tool to grasp bowel tissue so that the tool that will not damage the bowel. Operations 612, 614 can configure an RSS 504_{C} to use machine vision to identify bowel tissue during a surgical procedure. In response to machine vision recognizing bowel tissue, the RSS 504_{C} can be configured to make an intra-operative recommendation of a tool suitable for use to grasp the bowel tissue.

As a further example, operations 612, 614 can configure RSS 504_{C} to constrain force imparted by a tool based upon tissue type. Different anatomical tissues have different properties. If the patient feature information indicates that delicate anatomical tissue, such as bowel tissue, is to be manipulated, then operations 612, 614 can configure the RSS 504_{C} to limit force imparted to delicate tissue to a lower force level. If the patient feature information indicates that anatomical tissue is to be removed or excised from the body, however, then operations 612, 614 can configure the RSS 504_{C} to permit grasping of that tissue with greater force. Operations 612, 614 can configure RSS 504_{C} to use machine vision to recognize when a tool is being used to grasp delicate tissue and when the tool is being used to grasp tissue to be removed and to constrain force imparted by the tool accordingly.

As a further example, operations 612, 614 can configure RSS 504_{C} to constrain force imparted to cancerous anatomical tissue. Pre-operative patient health information for a prostatectomy procedure may indicate whether prostate tissue is cancerous. In general, cancerous prostate tissue is handled with special care to minimize damage to the prostate capsule to avoid release and spread of cancerous tissue. Operations 612, 614 can configure RSS 504_{C} to constrain force imparted to a prostate capsule during a prostatectomy procedure. The RSS 504_{C} can be configured to allow less force to be imparted to a prostate capsule if patient health information indicates that it is cancerous than if the patient health information indicates that it indicates that it is non-cancerous. Operations 612, 614 can configure RSS 504_{C} to use machine vision to recognize when a tool is being used to grasp a prostate capsule so that force can be automatically constrained accordingly.

As a further example, a surgeon may pre-operatively indicate a preferred suture to be used during a current surgical procedure. A needle driver tool selection and needle driver surgical technique may depend upon suture selection. For example, a braided suture requires special suturing technique to avoid damage to the suture. Operations 612, 614 can configure RSS 504_{C} to use machine vision to determine when a surgeon is engaging in a suture operation and to identify the kind of suture in use and to provide an intra-operative video visible via displays 34, 36 to demonstrate proper technique for manipulating a braided suture, for example.

As a further example, operations 612, 614 can configure RSS 504_{C} to set energy levels for a cauterization tool based upon patient tissue type. Resection of tissue typically results in bleeding. A cauterization tool uses energy to cause coagulation. Different energy levels are suitable to cause coagulation at different tissue types. For example, lower energy often is used to coagulate bowel tissue than to coagulate liver tissue.

### C. Recorded Surgical Procedure Data Sets:

Table 1 represents an example recorded surgical procedure data set, e.g., surgical data set, e.g., 505₁.

TABLE 1

Surgical Procedure Features
Patient Health Information
Surgeon Skill Features
Tool Set Features
Surgical Outcome Features

In the illustrated embodiment, the tool set recommendation engine 502 receives any number of surgical procedure data sets 505₁-505ₙ, which are then stored in a non-transitory computer readable storage medium, with each set corresponding to a previously performed instances of a surgical procedure. The data within the surgical procedure data sets may correspond to any feature of a corresponding surgical procedure. More particularly, a surgical procedure data set may refer to any data that were input into, measured, recorded, or otherwise gathered/obtained by a surgical system in association with a surgical procedure that was performed using the robotic surgical system. The data of a surgical procedure data set may be input to a robotic surgical system before a surgical procedure (e.g., manually or by any form of data transfer) and/or may be gathered, measured, recorded, or otherwise obtained by the robotic system during the procedure. The surgical procedure data sets may include robotic system information about a surgical procedure that includes kinematics information indicative of position and/or motion of manipulator support structures 72 and support spar 74 of a manipulator system 22 during a surgical procedure and/or may include actuation states of tools 26 and moveable components 428 during a surgical procedure. The surgical procedure data sets may include robotic system information about a surgical procedure such as endoscopic setup, sensor setup, tool registration information, table angle on which a patient rests, for example. Surgical procedure data obtained during a surgical procedure may be in the form of log files and also may include other data corresponding to the procedure.

In an example embodiment, each surgical procedure data set corresponds to a single surgical procedure performed using a robotic surgical system e.g., one of surgical systems 504₁-504ₙ. One or more surgical procedure data sets may be collected from each one of the robotic systems 504₁-504ₙ, for example. Surgical procedure data collected from a respective robotic surgical system may correspond to one or more types of surgical procedure performed using that robotic surgical system.

Surgical procedure data sets may include information concerning a surgeon's demonstrated robotic skills during a surgical procedure as measured during the surgical procedure. The measurements of surgeon skill may include measures of surgeon's efficiency during the procedure (e.g., duration of the procedure and/or amount of time a patient was under anesthetic) and surgical outcome, measurements of surgeon skill may include information indicative of consumption (e.g., efficiency of use) of surgical supplies and effectiveness of surgical tools used/selected.

In an example embodiment, surgical procedure data sets identify both tools and surgical events in which the tools are used. The relationship between tools and events can be complex and the surgical procedure data sets reflect this complexity. Numerous potential combinations of surgical events and surgical tools used during the events may be recorded in surgical procedure data sets, such as one or more of: suturing, involving use of a needle driver, mega needle driver or suture cut needle driver and suture line; retraction of tissue involving use of bowel grasper, pro-grasp, tip-up retractor; ligation, involving use of needle driver, stapler, vessel sealer extend or synch or seal; cauterizing involving use of Fenestrated bipolar, Maryland bipolar, vessel scaler extend, Monopolar scissor, monopolar hook, synchroscal; undermining involving use of; ablating tissue involving, use of monopolar spatula, monopolar hook, monopolar scissors; incising tissue, involving use of scissors, stapler, monopolar scissors, hook, spatula; blunt dissection involving use of Maryland bipolar, suction irrigator, fenestrated bipolar; sharp dissection, involving use of monopolar scissors, spatula, scalpel; removing tissue from the surgical environment, involving use of pro-grasp, Maryland bipolar, fenestrated bipolar, cardier; applying a clip; applying a clamp; applying a grasper involving use of pro-grasp, fenestrated bipolar, cardier, double fenestrated grasper, tenaculum. Referring to Table 1, data associated with surgical procedures performed at the one or more RSSs 504₁-504ₙ may be saved in several categories similarly to that of Table 1. The example surgical procedure data set includes surgical procedure features that indicates characteristics of a corresponding surgical procedure represented by the instance such a surgical type, surgical stages and surgical events occurring during the surgical procedure. The example surgical procedure data set includes patient health features that indicate health characteristics of a patient who was the subject of the corresponding surgical procedure. The example surgical procedure data set includes surgeon skill level information that indicates skill level features of a surgeon who performed the corresponding procedure. The example surgical procedure data set includes tool set information that indicates tool set features suitable for a surgical event type and a patient health characteristic such as tool size, for example. The example surgical procedure data set includes surgical outcome features of the corresponding surgical procedure.

A surgical procedure data set may identify different surgical procedure events such as, suturing, retraction of tissue, stapling tissue, cauterizing, vessel sealing, ablating tissue incising tissue, blunt dissection, suction, irrigation, sharp dissection, applying a clip, applying a clamp, and applying a grasper, for example. A surgical procedure data set may identify different robotic system setting or performance information during identified stages or events during an identified surgical procedure such as, kinematics information, endoscope setup and sensed images, sensor setup and sensor measurements, and tool registration information, tool speed scaling information, and table angle. For example, a surgical procedure data set may identify a hierarchy of surgical procedure information associated with surgical system information. The surgical procedure feature information can identify a surgical procedure and indicate the tools to be used in each stage. An example RSS 504_{C} includes three arms that may be identified as left, right, and center. A surgical procedure data set may indicate which tools are recommended to be used at which arm locations during each stage of a surgical procedure

Referring to patient health features, a surgical procedure data set may identify various features of a patient who is the subject of a corresponding surgical procedure. Patient health information can include height, weight, body mass index, treatment (e.g., medication), patient anatomical features such as those related to key artery veins, Ureter or nerves, bile duct. Tumor size and location, adhesions, diverticulitis, endometriosis, bleeding, for example may also be included. Many of a patient's anatomical features may be identified pre-operatively such as through imaging such as CT scan, x-ray, or MRI, for example. Other patient anatomical features may be identified intra-operatively by a surgeon or through automated image analyses based on images captured by an imaging device. Pre-operative patient health information may be manually entered by a user, uploaded from a previously existing image file (e.g., CT scan, x-ray, or MRI) or text a file, received from a patient monitoring device, or communicated in any other manner. Examples of intra-operatively identified patient features may include tissue thickness, a patient's neural or cardiac activity as measured by a patient monitoring device, and intra-operative images of anatomical structures captured using an endoscopic imager.

Referring to surgeon skill features, a surgical procedure data set may identify information related to a general skill-level and robotic tool control skills of a surgeon who performed a corresponding surgical procedure. Robotic tool control skill level information may include skill-level parameters such as the number of robotic surgical procedures completed by the surgeon, detailed information about specific types of robotic surgical procedures completed by the surgeon, medical training, and robotic tool control skills such as tool-specific experience with a particular tool or a group of related tools. Different robotically controlled surgical tools can require different robotic tool control skills. The surgeon skill level information may identify a surgeon skill level of a peer group in which a surgeon who performed the corresponding surgical procedure was a member. For example, a first peer group skill level may include surgeons having a high level of general surgical experience and a lower robotic skill level. A second peer group skill level may include surgeons having a medium level of general surgical experience but intermediate robotic skill level. A third peer group skill level may include surgeons currently practicing at an academic hospital institution and having at least an intermediate robotic skill level. Indicia of general surgical experience can include information such as number of surgeries performed, and/or certifications, for example. Hence, the effectiveness of a surgeon at using a robotically controlled surgical tool during a surgical procedure can depend not only upon the surgeon's general level of experience as a surgeon, but also upon the level of the surgeon's robotic tool control skills.

Table 2 identifies several examples of distinct core sets of robotic tool control skills that are useful during a robotic surgical procedure.

TABLE 2

Surgical Skill
Bimanual wrist manipulation
Camera Control
Master clutching to manage hand position
Use of a third instrument arm
Activating an energy source
Appropriate depth perception
Awareness of forces applied by instruments

In some embodiments, surgical procedure data set may rate surgical skill level of a surgeon for a robotic tool control skill category as novice, intermediate, or experienced. A surgical procedure data set may indicate that a surgeon's skill level varies from one skill category to the next and varies from one surgical instrument to the next. The skill assessment scale is generally applicable to any robotically assisted surgical procedure, regardless of surgical specialty. Activities during a surgical procedure can require a combination of surgical tools. For instance, neurovascular bundle dissection (nerve sparing), often involves use of multiple surgical tools such as a prograsper and robotic scissors, clip appliers, and suturing to ligate vessels. Another example activity can include Sleeve Gastrectomy and a corresponding surgical fool could include proper staple loads. Moreover, different surgical activities often require combinations of multiple robotic tool control skills. For example, performance of a continuous suturing surgical activity can require the following combination of surgical skills: instrument wrist manipulation, needle grasping, needle passing and orientation between two instruments, tissue grasping, and needle driving.

Referring to tool set information 608, a surgical procedure data set may identify tools used during a surgical procedure. For example, a tool set may include one or more of a needle driver, a mega needle driver or suture cut needle driver and a suture line. A tool set may include a bowel grasper, pro-grasp, tip-up retractor, a stapler, vessel sealer extend or synch or seal. A tool set may include a bipolar or monopolar cauterization tool, Maryland bipolar, Monopolar scissor, or monopolar hook. A tool set may include a synchroscal, a monopolar spatula, a monopolar hook, a monopolar scissors, a suction irrigator, a pro-grasp, a fenestrated bipolar, clips, clamps a double fenestrated grasper, or tenaculum. A tool set may include a flat end endoscope, 30-degree slanted end endoscope (for an angled view).

Tool set information also may indicate constraint upon use of a tool during a surgical procedure. For example, tool set information may indicate recommended actuation state of a tool. For instance, tool set information may indicate a tool's speed of operation during a surgical procedure such as high, medium, or low speed operation. Tool set information may indicate a position constraint imposed upon a tool during a surgical procedure such as a minimal distance from other tools or a minimal distance from an anatomical structure, for example. Tool set information may indicate a power usage constraint imposed upon a tool during a surgical procedure such as maximum power or a maximum time of energization during cauterization, for example.

As to surgical outcome information 710, a surgical procedure data set may identify outcome information such as clinical patient outcome and efficiency/waste in use of surgical implements. Operating rooms generally are stocked with only the tools that are necessary for safety and for a surgical procedure. Other tools that are not required are stored within inventory separate from the operating room. Medical staff may be required to pre-operatively obtain surgical tools from inventory that are needed for a procedure and to post-operatively return unused tools to inventory after the procedure. Moreover, surgical generally tools cannot be re-used after they are removed from sterilized packaging and must instead be discarded, whether or not they are actually used in a surgical procedure. The outcome information can be used as a basis to make decisions as to what tool resources to bring into the operating room for a procedure and what tools to unpackage before a procedure. These decisions can reduce staff time in returning unused tools to or obtaining unnecessary tools from inventory and can avoid unnecessarily unpackaging tools that are not actually used. For example, a surgeon may request three staple loads for use in a surgical stapler during a procedure, but the surgical outcome information 710 may indicate that only two staple loads typically are used. Based upon this information, medical staff may bring three staple loads to the operating room but only unpackage two of the staple loads. Alternatively, a surgeon may provide no preference as to number of staple loads. However, based upon the surgical outcome information 710 indicating that two staple loads typically are used, the medical staff may bring two staple loads to the operating room.

### D. Current Surgical Procedure Data:

Table 3 represents an example current surgical procedure data set.

TABLE 3

Surgical Procedure Features
Patient Health Features
Surgical Skill Features

The example current surgical procedure data set includes current surgical procedure information that indicates features of a corresponding current surgical procedure such as surgical procedure name, stages, and anticipated events. The current surgical procedure information can include the same kinds of surgical procedure information that is included in the recorded surgical procedure data sets. The current surgical procedure data set includes patient health information that can include pre-operative information such as patient height, weight, body mass index, treatment (e.g., medication), patient anatomical features such as those related to key artery veins, ureter or nerves, bile duct., tumor size and location, adhesions, diverticulitis, endometriosis, bleeding. The patient health information 804 also can include intra-operative information obtained during a current surgical procedure. In some surgical circumstances, the appropriate surgical tool to recommend may be contingent upon intra-operative developments. For example, environmental events may be detected during a surgical procedure such as excessive smoke during cauterization or excessive bleeding. Moreover, an anatomical feature, such as an unexpected tumor, may be first detected during a surgical procedure. During a current surgical procedure, images from a surgical site internal to a patient's anatomy, sensor input received from the surgical site, or audio or other signals provided by a surgeon may be based upon in generating an intra-operative tool recommendation. The patient health information can include the same kinds of patient health information that is included in the recorded surgical procedure data sets. The example current surgical procedure data set includes surgeon skill level information that indicates skill level features, including those described above, for a surgeon who performs the current surgical procedure. The current surgeon skill levels can include general surgical skill levels and/or robotic surgical skill levels of the kinds described above for the recorded surgical procedure data sets. The current surgical procedure data set 800 also includes outcome information 808. Outcome information for a surgical procedure data set may include an outcome rating that indicates degree of salubriousness of the outcome for a patient and a surgeon's efficiency/wastefulness in consuming surgical instrument consumables

### E. Machine Learning-Based Operation of Tool Set Recommendation Engine

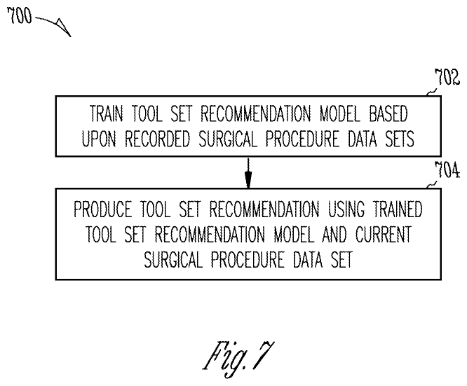

FIG. 7 is an illustrative flow diagram representing a machine learning-based method 700 of operating the tool set recommendation engine 502. Operation 702 trains a tool set recommendation model based upon recorded surgical procedure data sets. Operation 704 produces a tool set recommendation using the trained model and a current surgical procedure data set and optionally, a surgeon's tool set preferences.

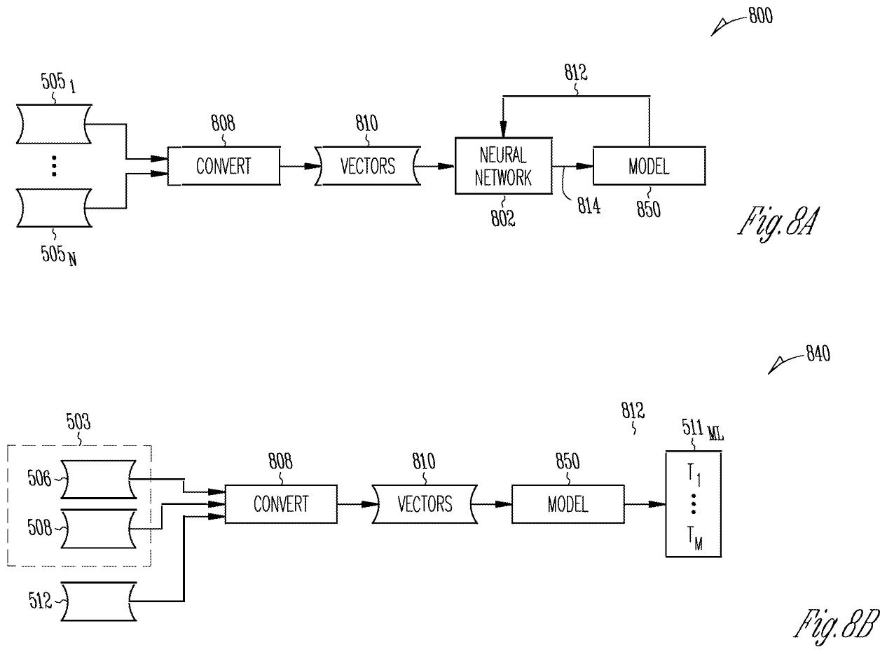

FIG. 8A is an illustrative drawing representing a method 800 of training a machine learning model 850. The method 800 provides additional details of the operation 702 of FIG. 7. FIG. 8B is an illustrative drawing representing a method 840 of using the trained machine learning model 850 to produce a tool set recommendation. The method 840 provides additional details of the operation 704 of FIG. 7. The example computer system 1600 of FIG. 13 can be configured to perform the example ML methods 800 and 840

Referring to FIG. 8A, the example ML model training method 800 employs machine learning to train an artificial neural network 802, based upon surgical procedure data sets 505₁ to 505ₙ that act as training data, to implement the ML model 850. Example training data features include surgical procedure information, patient health information, surgeon skill level information, and tool set information, and surgical outcome information, explained with reference to Table 1. Operation 808 transforms the training data, e.g., surgical procedure data sets 505₁ to 505ₙ, to an input vector representation 810 suitable for input to an input layer of the neural network 802. Edges 812, 814 represent an iterative training process in which the neural network 1002 is used to train the model 850.

Referring to FIG. 8B, the trained ML model 850 is used to produce a tool set recommendation 511_{ML} based upon a received current surgical procedure data 503, (which may include pre-operative data and intra-operative data 508), and optionally, also based upon user preference data 512. The trained ML model 850 can run before a current surgical procedure to determine tools set recommendations based upon data obtained pre-operatively. The trained ML model 850 also can run during a current surgical procedure to make updated tool set recommendations based upon data obtained intra-operatively. An example machine learning-based tool set recommendation 511_{ML} can include a plurality of tool identifiers (e.g., T₁ to Tₘ). An example trained ML model 850 can produce more than one tool suitable for use for a given activity during a current surgical procedure. For example, two kinds of staplers may be recommended. The two recommended staplers may have different weights associated with them. A surgeon may select among tools using tool set selection information 514, for example.

### E. Pattern-Based Operation of Tool Set Recommendation Engine

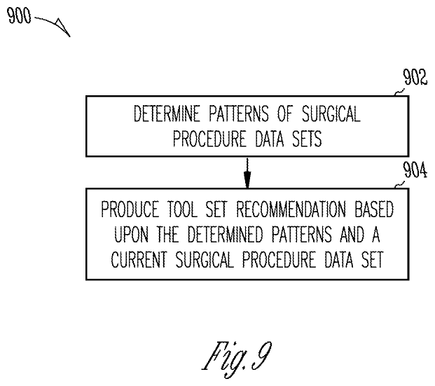

FIG. 9 is an illustrative flow diagram representing an example pattern-based method 900 of operating the tool set recommendation engine 502. Operations 902 and 904 of FIG. 9 correspond to and represent additional details of a pattern-based implementation of operation 606 of the method 600 of FIG. 6.

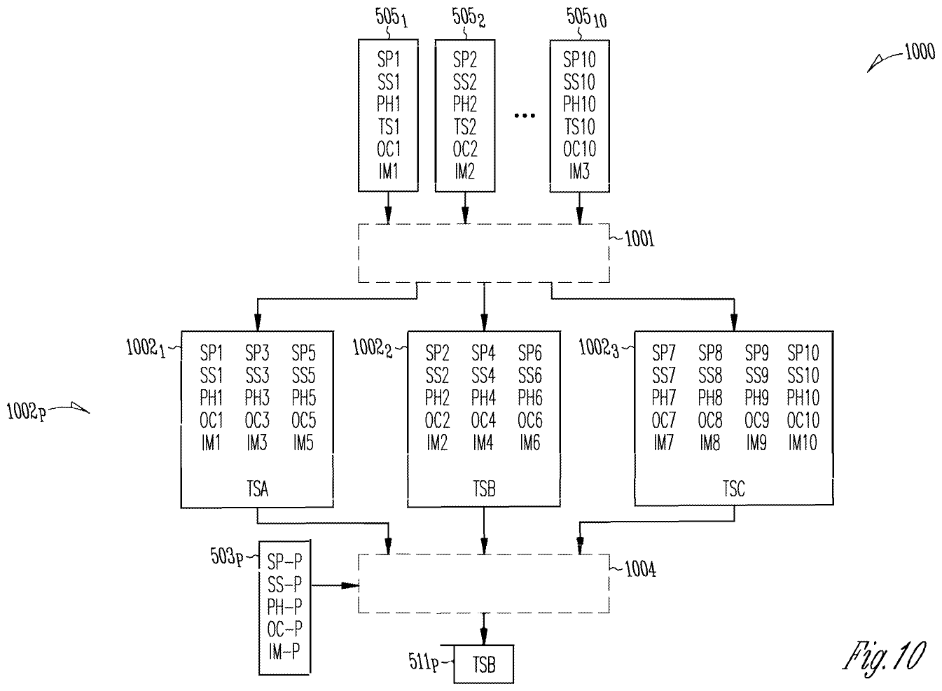

FIG. 10 is an illustrative data flow diagram representing an example use of the pattern-based method 1000. Recorded surgical procedure data sets 505₁-505ₙ are received at the tool set recommendation engine 502, where n=10. For economy of disclosure, the data sets include only surgical procedure (SP) information, surgeon skill (SS) level information, patient health (PH) information, tool set (TS) information, outcome (OC) information, and image (IM) information. Moreover, for economy of disclosure, details are not shown for the surgical procedure (SP), surgeon skill level (SS) information, patient health (PH) information, tool set (TS) and outcome (OC) information. An example first surgical procedure data set 505₁ includes first surgical procedure information SP1, first surgeon skill level information SS1, first patient health information PH1, first tool set TS1, and first surgical outcome information OC1. An example second surgical procedure data set 505₂ includes second surgical procedure information SP2, second surgeon skill level information SS2, second patient health information PH2, second tool set TS2, and second surgical outcome information OC2. An example tenth surgical procedure data set 505₁₀ includes tenth surgical procedure information SP10, tenth surgeon skill level information SS10, tenth patient health information PH10, tenth tool set TS10, and tenth surgical outcome information OC10.

Referring to FIGS. 9-10, during operation 1002, the tool set recommendation engine 502 determines surgical procedure data set patterns. More particularly, in an example embodiment of the tool set recommendation engine 502, operation 1001 uses one or more processors to execute an algorithm stored in a non-transitory computer readable storage medium to determine example surgical procedure data set patterns 1002_{P} (e.g., 1002, 1002₂, and 1002₃) based upon the received surgical procedure data sets 505₁-505₁₀. An example operation 1102 uses a statistical analysis process to make the determination. More particularly, in the illustrative example of FIG. 10, operation 1001 maps the surgical procedure data sets 504₁-504₁₂ to patterns 1002₁, 1002₂, and 1002₃, which are stored in a non-transitory computer readable storage medium. An example first pattern 1002₁ relates SP1, SS1 PH1, OC1, and IM1 from data set 504₁, SP3, SS3, PH3, OC3, and IM3 from data set 5043, and SP5, SS5, PH5, OC5, and IM5 from data set 504₅ to a first common tool set TSA. An example second pattern 1002₂ relates SP2, SS2, PH2, OC2, IM2 from data set 504₂, SP4, SS4, PH4, OC4, and IM4 from data set 5044, and SP6, SS6, PH6, OC6, and IM6 from data set 5046 to a second common tool set TSB. An example third pattern 1002₃ relates SP7, SS7, PH7, OC7, and IM7 from data set 5047, SP8, SS8, PH8, OC8, and IM8 from data set 504₈, SP9, SS9, PH9, OC9, and IM9 from data set 5049, and SP10, SS10, PH10, OC10, and IM10 from data set 504₁₀ to a third common tool set TSC.

As used herein, a “common tool set” refers to a tool set that is associated with a pattern and determined based upon tool sets of data sets included within the pattern. A common tool set can include alternative tool selections and can include guidance as to selecting among alternate tool selection possibilities. A common tool set can include options within different individual tools. For example, a tool set may include a staple load to include within a stapler, which may depend upon patient anatomy and the anatomical structure to be stapled. An example surgical procedure may include multiple surgical stages. Different surgical tools may be required during different surgical stages. In an example robotic surgical system 10, a sequence of surgical stages can be tracked manually or automatically, such as by using a computer processing system 27 based upon sequence of tools mounted to a spar 74 for use by the robotic surgical system 10. A change in surgical tools coupled to the surgical robotic system can indicate a change in surgical stage. Each of the example common tool sets TSA, TSB, TSC can include information indicating at which stages different tools are to be used. Referring to FIGS. 9-10, during operation 1004, the tool set recommendation engine 502 produces a tool set recommendation based upon a current surgical procedure data set and the determined surgical procedure data set patterns. Operation 1004 can run before a current surgical procedure to determine tools set recommendations based upon data obtained pre-operatively. Operation 1004 also can run during a current surgical procedure to make updated tool set recommendations based upon data obtained intra-operatively.

More particularly, in an example embodiment of the tool set recommendation engine 502, operation 1004 the tool set recommendation system 502 is configured with instructions stored in a non-transitory computer readable storage medium to cause one or more processors to execute an algorithm to determine a recommended common tool set based at least in part upon the developed patterns 1002₁, 1002₂, and 1002₃ and a current surgical procedure data set 503_{P}. An example operation 1004 can be configured to determine a recommended common tool set based upon comparing individual patterns 1002₁, 1002₂, and 1002₃ and the current surgical procedure data set 503_{P}. More particularly, an example operation 1004 can be configured to use a statistical analysis process to make the determination. It is contemplated that tool set recommendations may be based at least in part upon surgeon peer group patterns represent best practice for one or more surgeon peer groups.

In the example shown in FIG. 10, operation 1004 recommends the second common tool set TSB, which is provided as a pattern-based tool set recommendation 511_{P}. Alternatively, however, operation 1004 can be configured to provide a “ranking” of tools. For example, a first ranked tool option may include a primary option relating to the operating surgeon's preference, a second ranked tool option may be based on various factors including peer preferences (e.g., what are similarly skilled surgeons using?), and third ranked tool option may be based upon best-practice data (e.g., what the best of the best surgeons are using and achieving the best outcomes and efficiency). Moreover, an example pattern-based tool set recommendation 511_{P} can include not only the recommended common tool set, e.g., TSB, but also, surgical procedure data set features included within the pattern, e.g., pattern 1002₂, associated with the recommended common tool set. Thus, a pattern-based tool set recommendation 511_{P} includes not only a tool set recommendation, but also surgical procedure data set features associated with the recommended tool set.

### F. Intra-Operative Tool Set Recommendation

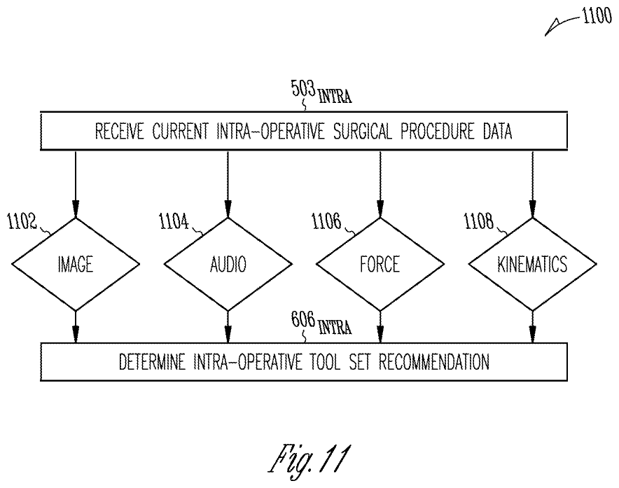

FIG. 11 is an illustrative flow diagram representing a method 1100 of operating the tool set recommendation engine 502 to use intra-operative surgical procedure data to produce an intra-operative tool set recommendation. The method 1100 can run before and during performance of the current surgical procedure. Operation 503_{INTRA} can use one or more sensors to receive intra-operative surgical procedure data during a current surgical procedure. An example surgical system 10 can include an imaging sensor 28, a microphone sensor 37, and/or a force sensor 432 to capture intra-operative information.

An example operation 1102 uses an image sensor coupled to an endoscope to capture images at a surgical site within a patient's anatomy. A computer processing subsystem 27 at a robotic surgical system 10 in which a current surgical is performed can be configured with executable instructions to perform image recognition of an anatomical feature and/or environmental feature of interest within one or more captured images. The image recognition determines an image identification to associate with the one or more captured images. The processing subsystem 27 can be configured with instructions to recognize within one or more images, an anatomical feature or other environmental feature of interest within an intra-operatively captured image data. An anatomical feature recognized within an image captured during the surgical procedure can be included within patient health feature of Table 3 during performance of the current surgical procedure to determine a tool recommendation based in part upon the one or more captured images.

An example operation 1104 uses a microphone sensor to capture audio information. The computer processing subsystem 27 can be configured with executable instructions to perform audio speech recognition to recognize verbal references to anatomical features and/or environmental features of interest to a surgeon. The processing subsystem 27 can be configured with instructions to use the speech to identify a descriptive label indicating an anatomical feature or other environmental feature of interest within an intra-operatively received audio communication data identification. An anatomical feature recognized based upon audio captured during the surgical procedure can be included within patient health feature information of Table 3 during performance of the current surgical procedure to determine a tool recommendation based in part upon the captured audio.

An example operation 1106 uses a tool's force sensor 432 to capture force information. The computer processing subsystem 27 can be configured with executable instructions to determine forces imparted to a surgical instrument that contacts tissue at a surgical site. The measured force can be included within patient health feature information in Table 3 during performance of the current surgical procedure to determine a tool recommendation based in part upon the captured audio.

An example operation 1108 uses sensors located to determine kinematics of the articulated support structures 73 support spar 74 and tool 410 to determine position information for a tool's movable component 428 and/or a wrist 430. The position information can be included within surgical procedure feature information in Table 1 during performance of the current surgical procedure to determine a tool recommendation such as a tool activation state or constraint to be implemented by the current RSS 504_{C}. based in part upon the position information. Operation of the current RSS 504_{C}, in use of a tool can be constrained based upon the recommended tool constraint.

### G. Display of Tool Set Recommendation and Related Guidance

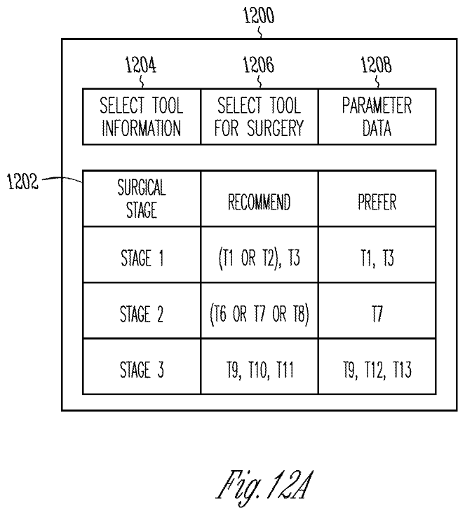

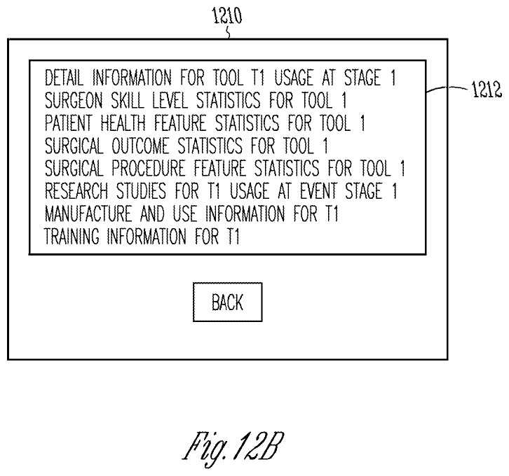

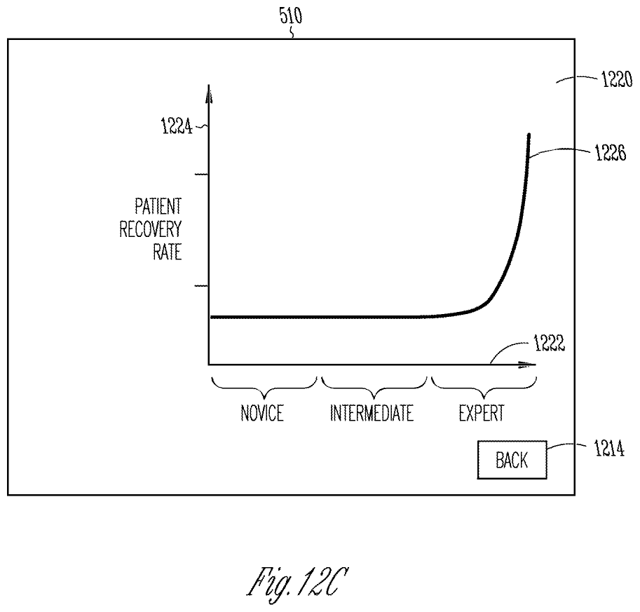

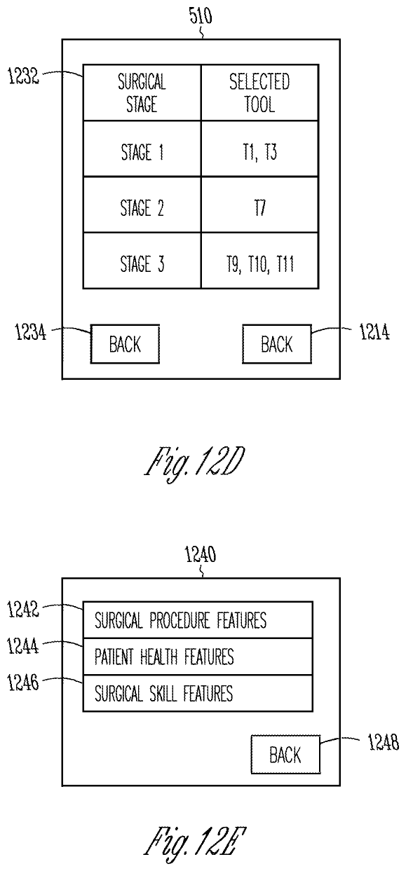

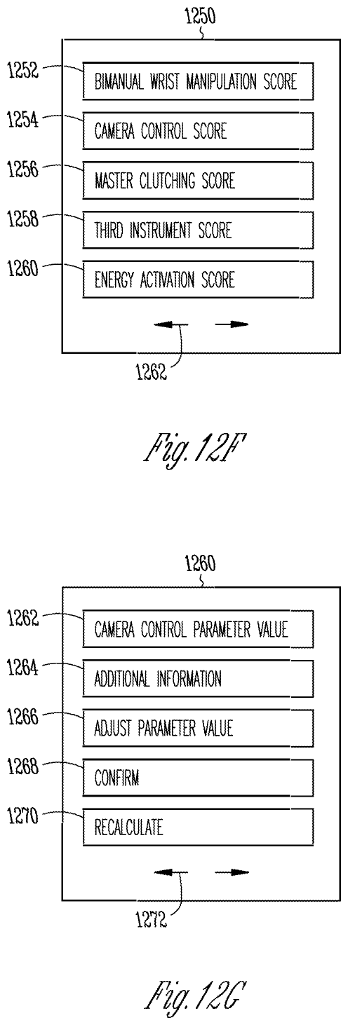

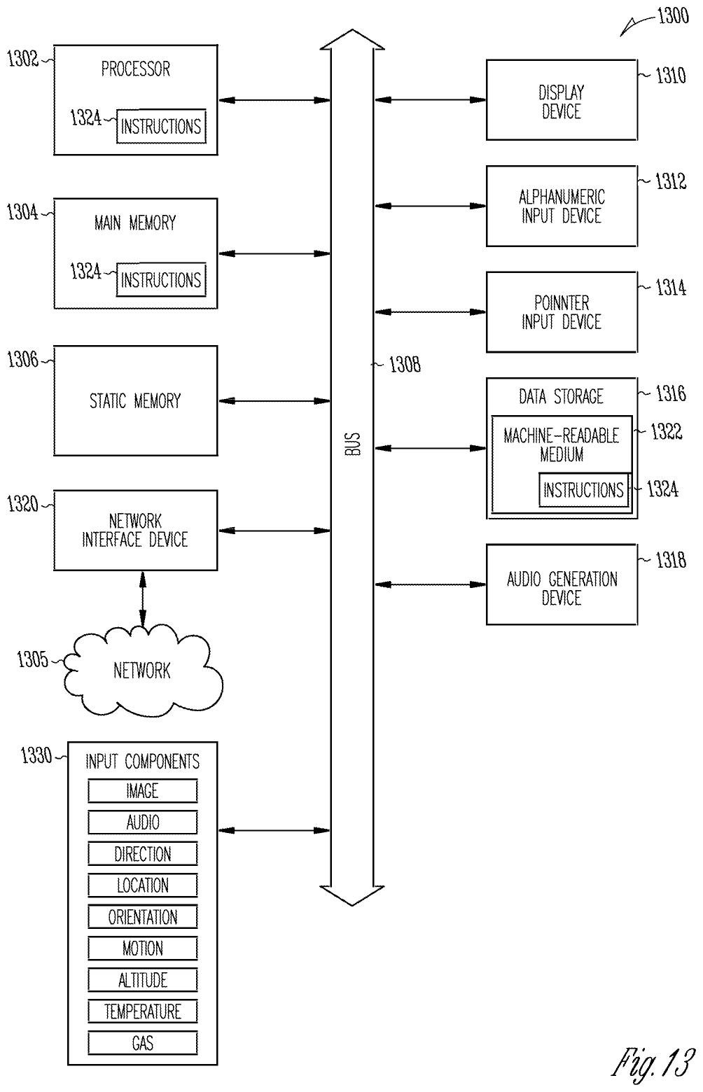

FIG. 12A is an illustrative drawing representing an example first user interface (UI) screen display 1200 produced at a display block 510 based upon recommendation information produced at line 511-1 and user preference information produced at lines 512-1, 512-2. The display block 510 is configured with instructions stored in a non-transitory computer readable storage medium to cause one or more processors to implement a display process to display UI screen displays. The example first UI screen display 1200 includes multiple display fields 1202 that indicate multiple example stages of an example surgical procedure and that indicate recommended tools and user-preferred tools for each of multiple example stages. An example surgical procedure includes three surgical stages: Stage 1, Stage 2, and Stage 3. An example user tool set recommendation block 502 is configured to produce a tool set recommendation for each stage as described with reference to FIG. 6.

Still referring to FIG. 12A, the recommendation block recommends use of either tool T1 or tool T2 plus the use of tool T3 during Stage 1; recommends use of tool T6 or tool T7 or tool T8 during Stage 2; and recommends use of tool T9, tool T10 or T11 during Stage 3. Also, for the illustrative example UI screen display 1200 displays user/surgeon's references as: tool T1 plus tool T3 during stage 1; tool T7 during Stage 2; and tools T9, T12, and T13 during Stage 3. It is noted that the tool set recommendations produced using the tool set recommendation block 510 include alternative tools that the user can select from. An example recommendation for Stage 1 recommends a selection between T1 and T2, and an example recommendation for Stage 2 recommends a selection from among T6, T7, and T8. The example first UI screen display 1200 includes a user-selectable first UI control input 1204 that can be used to access tool information about one or more of the tools indicated in the UI screen display 1200. The example first UI screen display 1200 includes a user-selectable second UI control input 1206 that can be used select one or more of the tools indicated in the UI screen display 1200 for use during the example surgical procedure. The example first UI screen display 1200 includes a user-selectable third UI control input 1208 to select a display of parameter data used to determine the tool recommendations set forth in the recommendation block.

FIG. 12B is an illustrative drawing representing an example second UI screen display 1210 produced at a display block 510 based upon user input at the example first display screen 1200. The illustrative example second display screen 1210 shows an example menu 1212 that indicates a selection of different kinds of information available about the first example tool T1. Each tool indicated in the first UI screen display 1200 is located at a different corresponding user-selectable field and that a user can navigate from the first UI screen display 1200 to the second UI screen display 1210 by selecting the first UI control input 1204 and user selectable field indicating a tool about which the user wishes explore more information. The example second UI screen display 1210 provides selectable information about a first tool T1. It is contemplated that user selection of the “Detail Information for Tool T1 usage at stage 1” causes the RSS 504_{C} to offer display of a training video as to use of T1 during stage 1 and to offer to the user a sign-up for simulation training on the use of tool T1. A user selects the example first UI control input 1204 and a selectable field at a region of the first UI screen display 1200 that displays a tool T1 indicator to cause the display process to produce the example second UI screen display 1210. The second UI screen display 1210 includes a Back selector input 1214 that a user can select to return to the first display screen 1200.

FIG. 12C is an illustrative drawing representing an example third UI screen display 1220 produced at a display block 510 based upon user input at the example second display screen 1210. The example third screen display 1220 shows an example graph indicating illustrative example surgical skill level statics for a selected example tool T1. The graph includes a first axis 1222 that indicates a range of skill levels from novice, to intermediate to expert and a second axis 1224 that indicates patient recovery rate. The graph contains an example curve 1226 that represents rate of recovery from a surgery in which a surgeon uses tool T₁ for a range of different surgeon skill levels. The illustrative curve 1226 of graph in the third UI screen display 1220 indicates that patients benefit significantly from the most skilled surgeons using T₁ and that both novice and intermediate surgeons achieve similar results, albeit far less beneficial than those achieved by the most skilled surgeons.

FIG. 12D is an illustrative drawing representing an example fourth UI screen display 1230 produced at a display block 510 based upon user input at the example first display screen 1200. The example fourth UI screen display 1230 includes multiple display fields 1232 that indicate the multiple example stages of the example surgical procedure and that indicate tools selected by a user for each of the multiple example stages. A user can navigate from the first UI screen display 1200 to the fourth UI screen display 1230. For example, a user selects the example second UI control input 1206 and selectable fields at regions of the first UI screen display 1200 that display tool indicators for tools that the user chooses to use during a surgery. In the example fourth UI screen display 1230, the user has selected tools T1 and T3 for use in Stage 1, has selected tool T7 for use in Stage 2, and has selected tools T9, T10, and T11 for use in Stage 3. The fourth UI screen display 1230 includes the Back selector input 1214 that a user can select to return to the first display screen 1200. The fourth UI screen display 1230 includes a Send selector input 1234 to send the indicated tool selection information over a network communications system to computers or other electronic devices operated by members of a surgical care team.

FIG. 12E is an illustrative drawing representing an example fifth UI screen display 1240 produced at a display block 510 based upon user input at the example first screen display 1200. The example fifth UI screen display 1240 includes multiple display fields 1242 that indicate example current feature sets used to determine the tool recommendations set forth in the tool recommendation block of the example first example screen display 1200. A user can select the example third UI control input 1208 to navigate from the first to the fourth screen display. The example fifth screen display 1240 provides a menu multiple selectable feature set inputs: surgical procedure features input 1242; patient health features input 1244; and surgical skill features input 1246. A user can select (e.g., click-on) a feature set input to navigate to a screen display corresponding to the selected input that provides corresponding feature set details that serve as a basis for the tool set recommendations listed on the first screen display 1200. It is contemplated, for example, that a user selection of the example surgical procedure features input 1242 causes navigation to a screen display that can identify one or more surgical events expected and/or planned during the current surgical procedure such as one or more of suturing, ligation, cauterizing, ablating tissue, cauterizing tissue, incising tissue, removing tissue from a surgical environment, sharp incision, applying a clip, applying a clamp. It is contemplated, for example, that a user selection of the example patient health features input 1244 causes navigation to a screen display that can identify one or more patient health features of a patient who is the subject of the current surgical procedure such as one or more of height, weight, body mass index, treatment (e.g., medication), artery features, vein features, ureter features, nerve features, bile duct features, tumor size and location, adhesions, diverticulitis, endometriosis, and bleeding. It is contemplated, for example, that a user selection of the example surgical skill features input 1246 causes navigation to a screen display that can identify skill level at various surgical tasks of a surgeon scheduled to perform the surgical procedure. Surgical skill level information can include one or more of the number of robotic surgical procedures completed by the surgeon, detailed information about specific types of robotic surgical procedures completed by the surgeon, medical training of the surgeon, and robotic tool control skills such as tool-specific experience with a particular tool or a group of related tools. Robotic tool control skill levels can include a bimanual wrist manipulation score, a camera control score, a master clutching score, a third instrument score, an energy activation score, a depth perception score, and a force awareness score. The surgeon skill level information may identify a surgeon skill level of a peer group in which a surgeon who performed the corresponding surgical procedure was a member. Additional indicia of general surgical skill level can include information such as number of surgeries performed, and/or certifications, for example. A Back control input 1248 is provided to return to the first UI screen display 1200

FIG. 12F is an illustrative drawing representing an example sixth UI screen display 1250 produced at a display block 510 based upon user input to select the surgical skill features input 1246 at the example fifth screen display 1240. The sixth UI screen display 1250 displays a selectable menu list of surgical skill features. It will be appreciated that not all surgical skill features can be displayed at once and the sixth UI screen display 1250 includes previous and next navigation arrows to navigate through the list. The example list segment in FIG. 12F includes the following selectable menu items robotic tool control skills features: bimanual wrist manipulation score item 1252, a camera control score item 1254, a master clutching score item 1256, a third instrument score item 1258, an energy activation score item 1260. A user can select a list item (e.g., by clicking on it) to access parameter value information associated with the selected item. The example sixth UI screen display 1250 also includes selectable forward and backward navigation arrows 1262

FIG. 12G is an illustrative drawing representing an example seventh UI screen display 1260 produced at a display block 510 based upon user input to select the camera control score feature at the example sixth screen display 1250. The seventh UI screen display 1260 includes a current parameter value field 1262, a selectable additional information control input 1264, a selectable parameter adjustment control input 1266, a confirmation control input 1268, a recalculate control input 1270, and navigation control input 1272, forward and backward control arrows. It is contemplated that the current parameter value field 1262 indicates a current value of the selected feature (the camera control score feature in this example) used in the determination of the tool set recommendations currently provided at the first UI screen display 1200 shown in FIG. 12A. It is contemplated that the selectable additional information control input 1264 provides additional information about the currently selected feature. It is contemplated that in this example, user selection of the additional information control input 1264 causes the RSS 504_{C} to offer display of a training video as to camera control and to also offer simulation training on camera control. It is contemplated that the selectable parameter adjustment control input 1266 can receive user input to adjust the current value of the parameter for the currently selected feature. For example, a user can change the camera score parameter value to reflect a recent improvement in camera movement skill level. It is contemplated that in the case of a numerical value, the selectable parameter adjustment control input 1266 can include an operative slider bar icon or an operative rotatable dial icon can be used to adjust the numerical score. Alternatively, it is contemplated that the selectable parameter adjustment control input 1266 can include a pull-down menu selection of adjustment options or can include a text entry field to receive adjustment information. It is contemplated that a user can select the confirmation control input 1268 to confirm an adjustment made using the selectable parameter adjustment control input 1266. It is contemplated that a user can select the recalculate control input 1270 to cause the tool set recommendation engine 502 to recalculate a tool set recommendation presented at the first UI screen display 1260 using an adjusted parameter value. The navigation control inputs 1272 permit forward and backward navigation among UI screens. Thus, a user/surgeon can provide updated feature parameter information to cause adjustment of recommendations tool set recommendation engine 502.

### H. Computing Machine

FIG. 13 illustrates components of a computing machine 1300, according to some example embodiments, that is able to read instructions from a machine-storage medium (e.g., a machine-readable storage device, a non-transitory machine-readable storage medium, a computer-readable storage medium, or any suitable combination thereof) and perform any one or more of the methodologies discussed herein. Specifically, FIG. 13 shows a diagrammatic representation of the machine 1300 in the example form of a computing device (e.g., a computer) and within which instructions 1324 (e.g., software, a program, an application, an applet, an app, or other executable code) for causing the machine 1300 to perform any one or more of the methodologies discussed herein may be executed, in whole or in part.

In alternative embodiments, the machine 1300 operates as a standalone device or may be connected (e.g., networked) to other machines. In a networked deployment, the machine 1300 may operate in the capacity of a server machine or a client machine in a server-client network environment, or as a peer machine in a peer-to-peer (or distributed) network environment. The machine 1300 may be a server computer, a client computer, a personal computer (PC), a tablet computer, a laptop computer, a netbook, a set-top box (STB), a personal digital assistant (PDA), a cellular telephone, a smartphone, a web appliance, a network router, a network switch, a network bridge, or any machine capable of executing the instructions 1324 (sequentially or otherwise) that specify actions to be taken by that machine. Further, while only a single machine is illustrated, the term “machine” shall also be taken to include a collection of machines that individually or jointly execute the instructions 1324 to perform any one or more of the methodologies discussed herein.

The machine 1300 includes a processor 1302 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 1304, and a static memory 1306, which are configured to communicate with each other via a bus 1307. The processor 1302 may contain microcircuits that are configurable, temporarily or permanently, by some or all of the instructions 1324 such that the processor 1302 is configurable to perform any one or more of the methodologies described herein, in whole or in part. For example, a set of one or more microcircuits of the processor 1302 may be configurable to execute one or more modules (e.g., software modules) described herein.

The machine 1300 may further include a graphics display 1310 (e.g., a plasma display panel (PDP), a light emitting diode (LED) display, a liquid crystal display (LCD), a projector, or a cathode ray tube (CRT), or any other display capable of displaying graphics or video). The machine 1300 may also include an input device 1312 (e.g., a keyboard), a cursor control device 1314 (e.g., a mouse, a touchpad, a trackball, a joystick, a motion sensor, or other pointing instrument), a storage unit 1316, a signal generation device 1318 (e.g., a sound card, an amplifier, a speaker, a headphone jack, or any suitable combination thereof), and a network interface device 1320.

The storage unit 1316 includes a machine-storage medium 1322 (e.g., a tangible machine-readable storage medium) on which is stored the instructions 1324 (e.g., software) embodying any one or more of the methodologies or functions described herein. The instructions 1324 may also reside, completely or at least partially, within the main memory 1304, within the processor 1302 (e.g., within the processor's cache memory), or both, before or during execution thereof by the machine 1300. Accordingly, the main memory 1304 and the processor 1302 may be considered as machine-readable media (e.g., tangible and non-transitory machine-readable media). The instructions 1324 may be transmitted or received over a network 1326 via the network interface device 1320.

In some example embodiments, the machine 1300 may be a portable computing device and have one or more additional input components (e.g., sensors or gauges). Examples of such input components include an image input component (e.g., one or more cameras), an audio input component (e.g., a microphone), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), and a gas detection component (e.g., a gas sensor). Inputs harvested by any one or more of these input components may be accessible and available for use by any of the modules described herein.

FIG. 14 illustrates an example method of recommending a tool for a computer-assisted robotic system, according to some embodiments. At least the tool set recommendation engine 502 can perform method 1400. In an aspect, the system can include one or more processors to at least partially perform the method 1400.

At 1410, the method 1400 can receive skill data and patient data. In an aspect, the skill data is associated with a medical procedure, and the patient data is associated with the medical procedure. For example, the skill data can be associated with a phase of a medical procedure, or a task of a phase of a medical procedure. For example, the patient data can be associated with a phase of a medical procedure, or a task of a phase of a medical procedure. Thus, the skill data and the patient data can each be associated with a given medical procedure, or task or phase thereof, to provide a technical improvement of highly granular tool recommendations beyond the capability of manual processes to achieve. In an aspect, the system can receive medical procedure information that indicates at least one of a stage or an event of the medical procedure, where the tool data can include a recommendation for the recommended tool during at least one of the stage or the event. For example, the system can receive the medical procedure information from the robotic system as one more robotic metrics indicative of a phase of the medical procedure, or a task of the phase.

At 1412, the method 1400 can receive the skill data indicative of a skill level for a surgeon. In an aspect, the skill data comprises at least one of an overall skill level of the surgeon, a procedure-based skill level of the surgeon associated with a type of the medical procedure, a task-based skill level of the surgeon associated with a type of task to be performed in the medical procedure, or an instrument-based skill level of the surgeon associated with the recommended tool. In an aspect, the skill level of the skill data indicates at least one of a peer group of one or more surgeons that can include the surgeon, surgical experience of the surgeon, experience of the surgeon with respect to the robotic system, or experience of the surgeon with respect to one or more tools associated with the skill data. For example, a peer group of one or more surgeons including the surgeon can correspond to a group of surgeons having an expert skill level indicative of a highest surgeon skill, an experienced skill level indicative of an intermediate skill level lower than the highest skill level, and a novice skill level indicative of a lowest skill level lower than the intermediate skill level and the highest skill level. For example, the surgical experience of the surgeon can correspond to a skill level of the surgeon having a tier or a quantitative value. For example, the surgical experience of the surgeon can be indicated by a quantitative value, and the skill level of the surgeon can be determined by identifying a skill level with one or more quantitative values or a range of quantitative values matching the surgical experience of the surgeon. The quantitative value for the surgical experience of the surgeon can correspond to a number of years, months, weeks, or days performing medical procedures, but is not limited thereto. For example, experience of the surgeon with respect to the robotic system can be indicated by a quantitative value, and can be correlated with a skill level at least as discussed above with respect to the surgical experience of the surgeon. The quantitative value for the experience of the surgeon with respect to the robotic system can correspond to a number of days, hours, or minutes using the robotic system, but is not limited thereto. For example, the experience of the surgeon with respect to one or more tools can be indicated by a quantitative value, and can be correlated with a skill level at least as discussed above with respect to the surgical experience of the surgeon. The quantitative value for the experience of the surgeon with respect to the robotic system can correspond to a number of days, hours, or minutes using the tool, but is not limited thereto. At 1414, the method 1400 can receive the patient data for a patient. At 1416, the method 1400 can receive the skill data and the patient data by a processor.

At 1420, the method 1400 can determine tool data indicative of a recommended tool. In an aspect, the system can select the recommended tool from among the one or more tools, according to the skill level of the surgeon or the peer group can include the surgeon corresponding to a first skill level. The system can select the recommended tool from among a subset of the one or more tools, according to the skill level of the surgeon or the peer group can include the surgeon corresponding to a second skill level. For example, the system can determine or identify one or more tools associated with a robotic system, or a robotic system and a given medical procedure. The system can identify whether one or more of the tools are associated with one or more skill levels, one or more quantitative values associated with a surgeon, peer group of a surgeon, or experience level at least as discussed herein. For example, the system can determine that all tools of a robotic system are associated with an expert skill level, that a first subset of all tools is associated with an experienced skill level, and that a second subset of the first subset of tools is associated with a novice skill level. Thus, the system can provide a technical improvement to select only those tools appropriate for the skill level of a given surgeon, and avoid recommendations for tools the surgeon is ill-prepared to use. In an aspect, the system can modify, based on preference data indicative of a preference of the surgeon, the tool data. For example, the system can identify one or more selections of one or more preferred tools associated with the surgeon, and can modify the tool data to increase recommendation values of tool data for the preferred tools. For example, the system can identify one or more selections of one or more less preferred tools associated with the surgeon, or identify one or more tools not selected as preferred tools, and can modify the tool data to decrease or exclude recommendation values of tool data for the less preferred tools or tools not selected as preferred tools.

In an aspect, the system can identify a plurality of peer groups comprising a first peer group and a second peer group, where a first set of tools are made available for recommendation to the first peer group, and a second set of tools are made available for recommendation to the second peer group. In an aspect, the system can map the surgeon to the first peer group of the plurality of peer groups. For example, mapping a first entity to a second entity as discussed herein can correspond to identifying an association between the first entity and the second entity. For example, the system can map the surgeon to the first peer group to determine or indicate that thee surgeon is associated with, or is part of, the first peer group. In an aspect, the system can identify, responsive to the mapping, that the first set of tools are available for recommendation to the surgeon. In an aspect, the system can select the recommended tool from the first set of tools that are available for recommendation to the first peer group.

In an aspect, the intra-operative data comprises at least one of one or more intra-operative images of an anatomy of the patient, intra-operative audio information, intra-operative information indicating occurrence of a predetermined event during the medical procedure, or intra-operative information indicating force imparted to a tool by an anatomical feature of the patient. In an aspect, the system can determine the tool data during medical procedure, using the model and based upon intra-operative kinematic state information corresponding to the robotic system. In an aspect, the system can determine, using the model and based on at least a portion of the patient data obtained before the surgical procedure, the tool data before the medical procedure.

At 1422, the method 1400 can determine the tool data using a model trained with machine learning. In an aspect, the system can train the model using machine learning, using input can include historical data corresponding to one or more instances of the medical procedure. In an aspect, the historical data can include at least one first feature indicative of respective outcomes of one or more of the instances of the medical procedure, and the historical data can include at least one second feature identifying surgical waste generated during the one or more of the instances of the medical procedure. At 1424, the method 1400 can determine the tool data using a model receiving as input the skill data. At 1426, the method 1400 can determine the tool data using a model receiving as input the patient data. At 1428, the method 1400 can determine the tool data by the processor. In an aspect, the system can identify, using a second model trained using machine learning to detect image features, a portion of the patient under manipulation by the portion of the robotic system.

The system can reconfigure, based on a property of the portion of the patient, the portion of the robotic system. In an aspect, the portion of the patient corresponds to tissue of the patient, and the property of the portion of the patient corresponds to delicate tissue. In an aspect, the system can train the model using machine learning based on one or more data sets indicative of a plurality of medical procedures, the one or more data sets can include corresponding patient data at least partially indicative of patient health, corresponding skill data, and corresponding tool data associated with the medical procedures. For example, reconfiguring the robotic system can correspond to automatically swapping one tool or instrument for another tool or instrument in the robotic system. The robotic system can activate one or more tools associated with a swap-in, and can deactivate one or more tools associated with a swap-out. Thus, the robotic system can automatically switch tools corresponding to recommendations for a given surgeon, surgeon peer group, medical procedure, phase, task, or any combination thereof, but is not limited thereto.

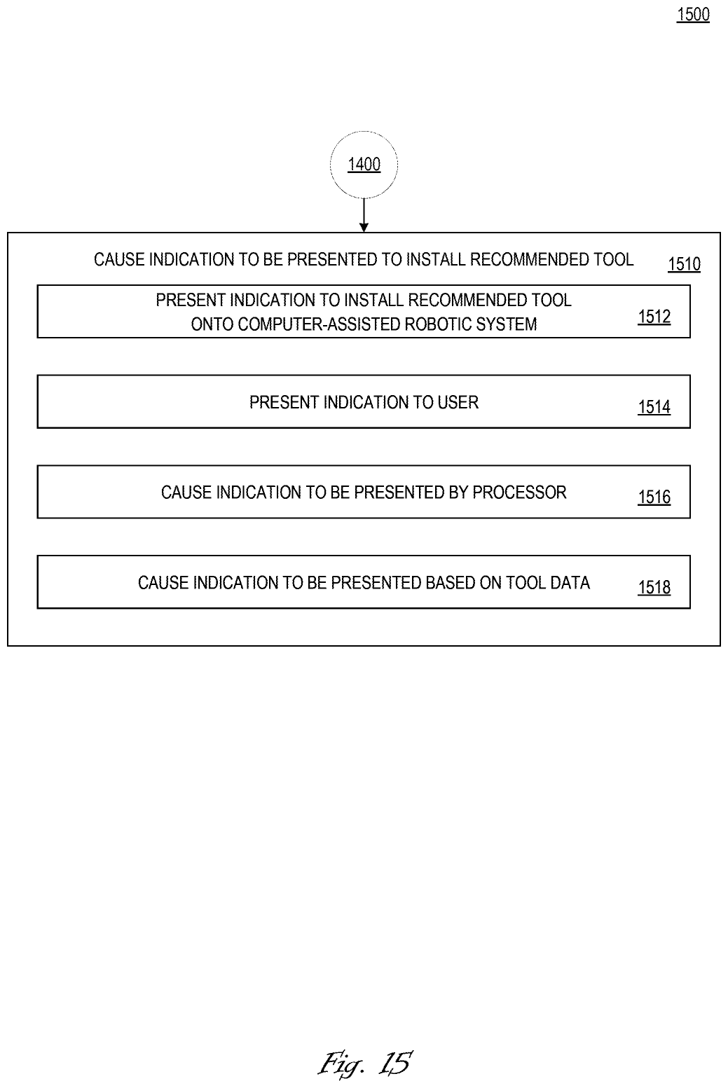

FIG. 15 illustrates an example method of presenting a recommendation for a tool for a computer-assisted robotic system, according to this disclosure. At least the tool set recommendation engine 502 can perform method 1500. In an aspect, the system can include one or more processors to at least partially perform the method 1500.

In an aspect, the system can capture intra-operative data during a medical procedure. The system can determine, using the model and based on the one or more intra-operative data, the tool data during the medical procedure. In an aspect, the system can detect, based on the intra-operative data, an environmental event during the medical procedure, where the environmental event corresponds to at least one of smoke during the medical procedure or bleeding during the medical procedure. For example, the environmental event can correspond to detection or identification of presence of smoke during the medical procedure. For example, the environmental event can correspond to detection or identification of presence of a level of smoke present during the medical procedure that satisfies a threshold indicative of an excessive level of smoke. For example, the level of smoke can correspond to a quantitative value that can be based on at least one of the tool, the robotic system, the medical procedure, or any combination thereof. For example, the environmental event can correspond to detection or identification of presence of bleeding during the medical procedure. For example, the environmental event can correspond to detection or identification of presence of a level of bleeding present during the medical procedure that satisfies a threshold indicative of an excessive level of bleeding. For example, the level of bleeding can correspond to a quantitative value that can be based on at least one of the tool, the robotic system, the medical procedure, or any combination thereof.

In an aspect, the system can receive pre-operative data captured during the medical procedure. The system can determine, using the model based on the pre-operative data, the tool data after the medical procedure. In an aspect, the pre-operative data comprises at least one of one or more intra-operative images of an anatomy of the patient, pre-operative audio information, pre-operative information indicating occurrence of a predetermined event during the medical procedure, or pre-operative information indicating force imparted to a tool by an anatomical feature of the patient. In an aspect, the system can determine, using the model and based on at least a portion of the patient data obtained during the medical procedure, the tool data during the medical procedure.

In an aspect, the system can receive, via a user interface, one or more preferences corresponding to the surgeon and indicative of one or more preferred tools associated with the surgical procedure. The system can receive, via the user interface, one or more selections of one or more of the preferred tools. The system can configure a robotic system according to the one or more selections. In an aspect, the system can automatically configure at least a portion of the robotic system in response to the one or more selections.

At 1510, the method 1500 can cause an indication to be presented to install the recommended tool. In an aspect, the system can provide a notification via an interface to install the recommended tool onto the computer-assisted robotic system. For example, the system can recommend, via a notification based on the tool data, to install the recommended tool onto the computer-assisted robotic system.

For example, the system can deliver a notification to a display as a graphical presentation. For example, the system can deliver the notification to a user interface associated with the surgeon, during the medical procedure. For example, the system can deliver the notification to a user interface associated with a medical staff member (e.g., a first assist) associated with the medical procedure, during the medical procedure. At 1512, the method 1500 can present the indication to install the recommended tool onto the computer-assisted robotic system. At 1514, the method 1500 can present the indication to a user. At 1516, the method 1500 can cause the indication to be presented by the processor. At 1518, the method 1500 can cause the indication to be presented based on the tool data. In an aspect, the system can reconfigure the robotic system to receive the recommended tool, or to constrain force imparted according to the recommended tool. In an aspect, the system can provide, based on the notification, an instruction to the robotic system to cause the robotic system to reconfigure at least a portion of the robotic system to receive the recommended tool. In an aspect, the system can determine, based on the skill level of the surgeon, a constraint on a force to be imparted by the recommended tool. In an aspect, the system can configure the robotic system to prevent the surgeon from imparting force, with the recommended tool, greater than or equal to the constraint. For example, the system can reduce the range of motion or the maximum magnitude at which force is applied. As discussed herein, to prevent the surgeon from imparting force is not limited to prevents all force from being applied, and can correspond to a reduction of force.

For example, reconfiguring the robotic system can correspond to providing a recommendation via a user interface to assist a medical staff member in swapping one tool or instrument for another tool or instrument in the robotic system. The robotic system can present one or more indications to guide the medical staff member to activate one or more tools associated with a swap-in. The robotic system can present one or more indications to guide the medical staff member to deactivate one or more tools associated with a swap-out. Thus, the robotic system can provide a user interface to switch tools corresponding to recommendations for a given surgeon, surgeon peer group, medical procedure, phase, task, or any combination thereof, but is not limited thereto, at a level of accuracy and responsiveness for a large number of robotic systems and tools, beyond the capability of manual processes to achieve.

### Executable Instructions and Machine-Storage Medium

The various memories (i.e., 1304, 1306, and/or memory of the processor(s) 1302) and/or storage unit 1316 may store one or more sets of instructions and data structures (e.g., software) 1324 embodying or utilized by any one or more of the methodologies or functions described herein. These instructions, when executed by processor(s) 1302 cause various operations to implement the disclosed embodiments.

As used herein, the terms “machine-storage medium,” “device-storage medium,” “computer-storage medium” (referred to collectively as “machine-storage medium 1322”) mean the same thing and may be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 1322 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage media, computer-storage media, and device-storage media 1322 specifically exclude carrier waves, modulated data signals, and other such media, at least some of which are covered under the term “signal medium” discussed below. In this context, the machine-storage medium is non-transitory.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer Readable Medium

The terms “machine-readable medium,” “computer-readable medium” and “device-readable medium” mean the same thing and may be used interchangeably in this disclosure. The terms are defined to include both machine-storage media and signal media. Thus, the terms include both storage devices/media and carrier waves/modulated data signals.

The instructions 1324 may further be transmitted or received over a communications network 1326 using a transmission medium via the network interface device 1312 and utilizing any one of a number of well-known transfer protocols (e.g., HTTP). Examples of communication networks 1326 include a local area network (LAN), a wide area network (WAN), the Internet, mobile telephone networks, plain old telephone service (POTS) networks, and wireless data networks (e.g., WiFi, LTE, and WiMAX networks). The term “transmission medium” shall be taken to include any intangible medium that is capable of storing, encoding, or carrying instructions 1324 for execution by the machine 1300, and includes digital or analog communications signals or other intangible medium to facilitate communication of such software.

In some embodiments, the network interface device 1312 comprises a data interface device that is coupled to one or more of an external camera 1330, an external microphone 1332, and an external speaker 1334 (e.g., external to the machine 1300). The camera 1330 may include a sensor (not shown) configured for facial detection and gesture detection. Any of the camera 1330, microphone 1332, and speaker 1334 may be used to conduct the presentation as discussed herein.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-storage medium or in a transmission signal) or hardware modules. A “hardware module” is a tangible unit capable of performing certain operations and may be configured or arranged in a certain physical manner. In various example embodiments, one or more computer systems (e.g., a standalone computer system, a client computer system, or a server computer system) or one or more hardware modules of a computer system (e.g., a processor or a group of processors) may be configured by software (e.g., an application or application portion) as a hardware module that operates to perform certain operations as described herein.

In some embodiments, a hardware module may be implemented mechanically, electronically, or any suitable combination thereof. For example, a hardware module may include dedicated circuitry or logic that is permanently configured to perform certain operations. For example, a hardware module may be a special-purpose processor, such as a field programmable gate array (FPGA) or an ASIC. A hardware module may also include programmable logic or circuitry that is temporarily configured by software to perform certain operations. For example, a hardware module may include software encompassed within a general-purpose processor or other programmable processor. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where the hardware modules comprise a general-purpose processor configured by software to become a special-purpose processor, the general-purpose processor may be configured as respectively different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over appropriate circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module implemented using one or more processors.

Similarly, the methods described herein may be at least partially processor-implemented, a processor being an example of hardware. For example, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an application program interface (API)).

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification may be presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

The above description is presented to enable any person skilled in the art to create and use systems and methods for recommending a tool set for a surgical procedure to be performed using a robotic surgical system. Various modifications to the embodiments will be readily apparent to those skilled in the art, and the generic principles defined herein may be applied to other embodiments and applications without departing from the spirit and scope of the invention. In the preceding description, numerous details are set forth for the purpose of explanation. However, one of ordinary skill in the art will realize that the embodiments in the disclosure might be practiced without the use of these specific details. In other instances, well-known processes are shown in block diagram form in order not to obscure the description of the invention with unnecessary detail. Identical reference numerals may be used to represent different views of the same or similar item in different drawings. Thus, the foregoing description and drawings of examples in accordance with the present invention are merely illustrative of the principles of the invention. Therefore, it will be understood that various modifications can be made to the embodiments by those skilled in the art without departing from the spirit and scope of the invention, which is defined in the appended claims.

1. A system for recommending a tool for a computer-assisted robotic system, comprising:
a non-transitory memory and one or more processors to:
receive skill data and patient data, the skill data indicative of a skill level associated with a surgeon, and the patient data corresponding to a patient;
determine, using a model trained with machine learning and receiving as input the skill data and the patient data, tool data indicative of a recommended tool; and
cause, based on the tool data, an indication to be presented to a user to install the recommended tool onto the computer-assisted robotic system.

2. The system of claim 1, wherein the skill data comprises at least one of:
an overall skill level of the surgeon;
a procedure-based skill level of the surgeon associated with a type of a medical procedure to be performed on the patient;
a task-based skill level of the surgeon associated with a type of task to be performed in the medical procedure; or
an instrument-based skill level of the surgeon associated with the recommended tool.

3. The system of claim 1, comprising the one or more processors to:
identify a plurality of peer groups comprising a first peer group and a second peer group, wherein a first set of tools are made available for recommendation to the first peer group, and a second set of tools are made available for recommendation to the second peer group;
map the surgeon to the first peer group of the plurality of peer groups;
identify, responsive to the mapping, that the first set of tools are available for recommendation to the surgeon; and
select the recommended tool from the first set of tools that are available for recommendation to the first peer group.

4. The system of claim 3, comprising the one or more processors to:
map the surgeon to the first peer group based at least in part on the skill level of the surgeon; and
input the first set of tools or an indication of the mapped first peer group into the model to determine the recommended tool.

5. The system of claim 1, comprising the one or more processors to:
capture intra-operative data during a medical procedure; and
determine, using the model and based on the intra-operative data, the tool data during the medical procedure.

6. The system of claim 5, comprising the one or more processors to:
detect, based on the intra-operative data, an environmental event during the medical procedure, wherein the environmental event corresponds to at least one of smoke during the medical procedure or bleeding during the medical procedure.

7. The system of claim 5, wherein the intra-operative data comprises at least one of one or more intra-operative images of an anatomy of the patient, intra-operative audio information, intra-operative information indicating occurrence of a predetermined event during the medical procedure, or intra-operative information indicating force imparted to a tool by an anatomical feature of the patient.

8. The system of claim 1, comprising the one or more processors to:
determine the tool data during a medical procedure performed on the patient, using the model and based upon intra-operative kinematic state information corresponding to the computer-assisted robotic system.

9. The system of claim 1, comprising the one or more processors to:
provide a notification via an interface to install the recommended tool onto the computer-assisted robotic system.

10. The system of claim 9, comprising the one or more processors to:
identify, using a second model trained using machine learning to detect image features, a portion of the patient under manipulation by a portion of the computer-assisted robotic system; and
reconfigure, based on a property of the portion of the patient, the portion of the computer-assisted robotic system to receive the recommended tool.

11. The system of claim 9, comprising the one or more processors to:
provide, based on the notification, an instruction to the computer-assisted robotic system to cause the computer-assisted robotic system to reconfigure at least a portion of the computer-assisted robotic system to receive the recommended tool.

12. The system of claim 9, comprising the one or more processors to:
determine, based on the skill level of the surgeon, a constraint on a force to be imparted by the recommended tool; and
configure the computer-assisted robotic system to prevent the surgeon from imparting force, with the recommended tool, greater than or equal to the constraint.

13. The system of claim 1, comprising the one or more processors to:
receive medical procedure information that indicates at least one of a stage or an event of the medical procedure, wherein the tool data includes a recommendation for the recommended tool during at least one of the stage or the event.

14. The system of claim 1, comprising the one or more processors to:
determine, using the model and based on at least a portion of the patient data obtained before a medical procedure is performed on the patient, the tool data before the medical procedure is performed on the patient.

15. The system of claim 1, comprising the one or more processors to:
determine, using the model and based on at least a portion of the patient data obtained during a medical procedure performed on the patient, the tool data during the medical procedure.

16. The system of claim 1, comprising the one or more processors to:
receive pre-operative data captured during a medical procedure performed on the patient; and
determine, using the model based on the pre-operative data, the tool data after the medical procedure.

17. The system of claim 16, wherein the pre-operative data comprises at least one of one or more intra-operative images of an anatomy of the patient, pre-operative audio information, pre-operative information indicating occurrence of a predetermined event during the medical procedure, or pre-operative information indicating force imparted to a tool by an anatomical feature of the patient.

18. The system of claim 1, comprising the one or more processors to:
receive, via a user interface, one or more preferences corresponding to the surgeon and indicative of one or more preferred tools associated with a medical procedure for the patient;
receive, via the user interface, a selection of at least one of the one or more preferred tools; and
configure a robotic system according to the selection.

19. The system of claim 18, comprising the one or more processors to:
automatically configure at least a portion of the robotic system in response to the selection.

20. The system of claim 1, comprising the one or more processors to:
modify, based on preference data indicative of a preference of the surgeon, the tool data.

21. The system of claim 1, comprising the one or more processors to:
train the model using machine learning based on one or more data sets indicative of a plurality of medical procedures, the one or more data sets including corresponding patient data at least partially indicative of patient health, corresponding skill data, and corresponding tool data associated with the plurality of medical procedures.

22. The system of claim 1, comprising the one or more processors to:
train the model using machine learning, using input including historical data corresponding to one or more instances of a medical procedure.

23. The system of claim 22, wherein the historical data includes at least one first feature indicative of respective outcomes of the one or more instances of the medical procedure, and the historical data includes at least one second feature identifying surgical waste generated during the one or more instances of the medical procedure.

24. A method for recommending a tool for a computer-assisted robotic system, comprising:
receiving, by a processor, skill data and patient data, the skill data indicative of a skill level associated with a surgeon, and the patient data corresponding to a patient;
determining, by the processor using a model trained with machine learning and receiving as input the skill data and the patient data, tool data indicative of a recommended tool; and
causing, by the processor and based on the tool data, an indication to be presented to a user to install the recommended tool onto the computer-assisted robotic system.

25. A non-transitory computer readable medium including one or more instructions stored thereon and executable by a processor to:
receive, by the processor, skill data and patient data, the skill data indicative of a skill level associated with a surgeon, and the patient data corresponding to a patient;
determine, by the processor using a model trained with machine learning and receiving as input the skill data and the patient data, tool data indicative of a recommended tool; and
cause, by the processor and based on the tool data, an indication to be presented to a user to install the recommended tool onto a computer-assisted robotic system.
